(19) United States
(12) Patent Application Publication
(10) Pub. No.: US 2024/0182541 A1
(43) Pub. Date: Jun. 6, 2024

(54) ANTIBODY PRODRUGS WITH CONSTANT DOMAINS

(71) Applicants: CONCEPT TO MEDICINE BIOTECH CO., LTD., Shanghai (CN); LEPU BIOPHARMA CO., LTD., Shanghai (CN)

(72) Inventors: Lei FANG, Shanghai (CN); Hao SHEN, Shanghai (CN); Peng ZENG, Shangahi (CN)

(21) Appl. No.: 18/582,563

(22) Filed: Feb. 20, 2024

Related U.S. Application Data
(63) Continuation of application No. PCT/CN2022/114310, filed on Aug. 23, 2022.

(30) Foreign Application Priority Data
Aug. 23, 2021 (WO) PCT/CN2021/114121

(51) Int. Cl. C07K 14/725; A61K 47/65; C07K 16/28

(52) U.S. Cl. CPC C07K 14/7051; A61K 47/65; C07K 16/2827; C07K 16/2863; C07K 2317/24; C07K 2317/31; C07K 2319/30

(57) ABSTRACT

Provided are antibody prodrugs that include an immunoglobulin superfamily constant region, such as CH3, or variant coupled to one or more chains of the antibody, optionally through a cleavable linker. It is discovered that the constant domain and variants serve as effective and safe masking moieties that inhibit the activity of the antibody. Once removed from the antibody, such as by a corresponding enzyme at a target treatment tissue, the antibody prodrug releases the active antibody. In tissues where no such enzyme is present, the antibody prodrug stays inactive, avoiding adverse effects in such tissues.

### BACKGROUND

Antibodies and antigen-binding fragments are commonly used in therapeutics, in particular for treating cancers. Despite their high specificity, however, these therapeutic agents can cause “on-target off-tumor” toxicities because the antigens or targets may be expressed in normal cells or tissue as well which might cause significant adverse effect. In these cases, high potency usually comes with high toxicity, which might limit the therapeutic window. Thus, there is an attempt to find an approach to widen the therapeutic window for these targets.

An antibody prodrug is a molecule that is inert but can be activated in a target diseased cell or tissue to generate an active antibody. An example antibody prodrug technology is the Probody™ technology platform developed by CytomX Therapeutics, Inc. In a Probody™ antibody prodrug, an IgG antibody, or a fragment thereof, is modified to include a masking peptide linked to the N-terminus of the light chain of the antibody through a protease-cleavable linker peptide. In the intact form, the antibody prodrug is effectively blocked from binding to the target antigen in healthy tissues. Once activated by appropriate proteases in the diseased environment, the masking peptide is released, releasing the active antibody for treating the disease.

Identification of a suitable masking peptide and corresponding linkers, however, have proven to be challenging.

### SUMMARY

It is discovered herein that a natural part of antibodies, such as the CH3 domain and the CH1/Cκ domains, can serve as an effective and safe masking moiety when fused to the N-terminus of an antibody (or antigen-binding fragment). Such a masking moiety significantly reduces, or even eliminates, the binding activity of the antibody. Once removed, the active antibody is released and regains its activity. The CH3-antibody fusion protein, therefore, serves as an antibody prodrug. The removal of the masking moiety may be achieved, e.g., by enzymatic digestion of a peptide linker that is included between the CH3 domain and the antibody. It is contemplated that other immunoglobulin superfamily constant regions, such as IgG CH3, IgG CH2, IgG CH1, IgG CL, and T-cell receptor (TCR) constant region, can also be used as the masking moiety, and such masking effect is applicable other variable regions, such as CH1, CH2, CL (kappa or lambda), and TCR variable region, as well. In addition, the masking moiety can be either conjugated to the variable region or fused together to form a fusion protein.

Accordingly, one embodiment of the present disclosure provides a molecule comprising (a) an immunoglobulin superfamily constant region or a fragment thereof covalently coupled to (b) an immunoglobulin superfamily variable region, wherein the variable region, when not coupled to the constant region, can bind to a target molecule, but the coupling of the constant region to the variable region inhibits such binding.

In some embodiments, the constant region (a) is fused to the N-terminus of the variable region or (b) is conjugated to the variable region. In some embodiments, the molecule does not include an immunoglobulin superfamily variable region on the N-terminal side of the immunoglobulin superfamily constant region.

In some embodiments, the constant region is selected from the group consisting of an IgG CH3, IgG CH2, IgG CH1, IgG CL, and a T-cell receptor (TCR) constant region, preferably CH3. In some embodiments, the variable region is selected from the group consisting of heavy chain variable region (VH), a light chain variable region (VL), and a T-cell receptor (TCR) variable region.

In some embodiments, the constant region, which is preferably CH3, is fused to the N-terminus of the variable region. In some embodiments, the molecule comprises a heavy chain variable region (VH), a first immunoglobulin superfamily constant region fused to the N-terminus of the VH, a light chain variable region (VL), and a second immunoglobulin superfamily constant region fused to the N-terminus of the VL, wherein the VH and VL collectively have binding specificity to the target molecule, and the first and second constant regions pair with each other. In some embodiments, the first and second constant regions are two CH3, a CH1 and a CL, or a TCR alpha chain and a TCR beta chain.

In some embodiments, the two constant regions are modified, as compared to the wild-type constant regions, to increase the heterodimerization of the masking moiety. In some embodiments, the two constant regions are modified, as compared to the wild-type constant regions, to include knob-in-hole substitutions, or charge-pair substitutions.

In some embodiments, the molecule does not include an additional immunoglobulin superfamily variable region on the N-terminal side of either the first or the second constant region. In some embodiments, the molecule does not include an additional immunoglobulin superfamily constant region on the N-terminal side of either the first or the second constant region.

In some embodiments, the molecule comprises: a first antigen-binding unit comprising a first VH paired to a first VL, a second antigen-binding unit comprising a second VH paired to a second VL, a first immunoglobulin superfamily constant region fused to the N-terminus of the first VH, a second immunoglobulin superfamily constant region fused to the N-terminus of the first VL, a third immunoglobulin superfamily constant region fused to the N-terminus of the second VH, and a fourth immunoglobulin superfamily constant region fused to the N-terminus of the second VL, wherein the first immunoglobulin superfamily constant region pairs with the second immunoglobulin superfamily constant region and inhibits the binding of the first antigen-binding unit, and the third immunoglobulin superfamily constant region pairs with the fourth immunoglobulin superfamily constant region and inhibits the binding of the second antigen-binding unit. In some embodiments, the first and second antigen-binding units can have the same sequence(s), target the same epitope or antigen, or target different epitopes or antigens.

In some embodiments, the first immunoglobulin superfamily constant region and the second immunoglobulin superfamily constant region are modified, as compared to the wild-type constant regions, to include knob-in-hole substitutions, or charge-pair substitutions, while the third immunoglobulin superfamily constant region and the fourth immunoglobulin superfamily constant region do not have the knob-in-hole substitutions, or the charge-pair substitutions.

In some embodiments, the third immunoglobulin superfamily constant region and the fourth immunoglobulin superfamily constant region have a pair of charge-pair substitutions or a pair of knob-in-hole substitutions, which substitutions are different from that between the first immunoglobulin superfamily constant region and the second immunoglobulin superfamily constant region.

In some embodiments, there are no more than 40 amino acid residues, preferably no more than 35, 30, 25, 24, 23, 22, 21 or 20 amino acid residues, and more preferably no more than 15, 14, 13, 12, 11, 10, 9, or 8 amino acid residues between T437, according to EU numbering (T468 according to Kabat numbering), of each CH3 domain and the N-terminus of the corresponding variable region.

In some embodiments, each CH3 domain is truncated to at least retain a fragment which is sufficient to inhibit the binding of the variable region to the target molecule, wherein the CH3 domain is preferably truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, C-terminal amino acid residue(s) as compared to the wild-type human IgG CH3 domain. In some embodiments, there are at least 8 amino acid residues, preferably at least 9, 10, 11 or 12 amino acid residues, and more preferably at least 13, 14, 15 16, 17, 18, 19 or 20 amino acid residues between T437, according to EU numbering (T468 according to Kabat numbering), of each CH3 domain and the N-terminus of the corresponding variable region.

In some embodiments, each CH3 domain is truncated to at least retain a fragment which is sufficient to inhibit the binding of the variable region to the target molecule, wherein the CH3 domain is preferably truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, N-terminal amino acid residue(s) as compared to the wild-type human IgG CH3 domain.

In some embodiments, each CH3 domain is fused to each variable region through a peptide linker, which is optionally cleavable, preferably enzymatically cleavable. In some embodiments, each enzymatically cleavable peptide linker is cleavable by an enzyme selected from the group consisting of fibroblast activation protein, urokinase-type plasminogen activator, matriptase, legumain, and a matrix metalloprotease. In some embodiments, each enzymatically cleavable peptide linker comprises an amino acid sequence selected from the group consisting of SEQ ID NO: 51-64 and 101-103.

In some embodiments, wherein each peptide linker is cleavable. In some embodiments, each peptide has identical sequence to one another.

In some embodiments, the constant region is conjugated to the variable region through a cleavable linker. In some embodiments, the cleavable linker is covalently attached to the side chain of an amino acid of the variable region. In some embodiments, the amino acid is located in the first framework region, the second framework region, the third framework region, the fourth framework region, or first CDR, the second CDR, or the third CDR. In some embodiments, the cleavable linker is capable of being cleaved by one or multiple proteolytic enzyme, protease or peptidase.

In some embodiments, each CH3 domain is of subclass IgG1, IgG2, IgG3 or lgG4.

In some embodiments, each CH3 domain comprises amino acid residues G371 through T437, according to EU numbering, of a full-length CH3 domain. In some embodiments, each CH3 domain comprises amino acid residues K360 through T437, according to EU numbering, of a full-length CH3 domain. In some embodiments, each CH3 domain comprises amino acid residues E345 through T437, according to EU numbering, of a full-length CH3 domain. In some embodiments, each CH3 domain comprises amino acid residues 31-97 of SEQ ID NO: 10, or amino acid residues 20-97, 10-97, 5-97, 4-97, 3-97, 2-97, or 5-101 of SEQ ID NO: 10. In some embodiments, one of the CH3 domains comprises amino acid residues 1-97 of SEQ ID NO: 19 and the other CH3 domain comprises amino acid residues 1-97 of SEQ ID NO: 20.

In some embodiments, the variable region is present in an antibody or fragment is a bispecific or trispecific antibody or fragment, each specificity comprising a variable region each of which is fused to or conjugated to an immunoglobulin superfamily constant region.

In some embodiments, the variable region is present in an antibody or fragment which is preferably a full-sized Fab antibody, a nanobody, a single-chain fragment, or a Bispecific T cell engager (BiTE).

Also provided, in one embodiment, is a fusion protein comprising a cleavable peptide linker fused to the C-terminus of an immunoglobulin superfamily constant region, wherein the fusion protein does not include an antigen-binding fragment on the N-terminal side of the immunoglobulin superfamily constant region.

In some embodiments, the fusion protein further comprises an immunoglobulin superfamily variable region fused to the C-terminus of the cleavable peptide linker. In some embodiments, the immunoglobulin superfamily constant region is selected from the group consisting of an IgG CH3, IgG CH2, IgG CH1, IgG CL, and a T-cell receptor (TCR) constant region, preferably CH3.

In some embodiments, there are no more than 40 amino acid residues, preferably no more than 35, 30, 25 or 20 amino acid residues, and more preferably no more than 15, 14, 13, 12, 11, 10, 9, or 8 amino acid residues, between T437, according to EU numbering (T468 according to Kabat numbering), of each CH3 domain and the C-terminus of the cleavable peptide linker.

In some embodiments, the CH3 domain is truncated to at least retain a fragment which is sufficient to inhibit the binding of the variable region to the target molecule, wherein the CH3 domain is preferably truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, C-terminal amino acid residue(s) as compared to the wild-type human IgG CH3 domain, or is truncated to remove at least one, preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, C-terminal amino acid residue as compared to the wild-type human IgG CH3 domain.

In some embodiments, the cleavable peptide linker is enzymatically cleavable, preferably cleavable by an enzyme selected from the group consisting of fibroblast activation protein, urokinase-type plasminogen activator, matriptase, legumain, and a matrix metalloprotease.

Also provided, in yet another embodiment, is a chimeric antigen receptor (CAR) that comprises the molecule of the present disclosure. Still further provided is a T-cell receptor (TCR) comprising one or more variable (V) regions and one or more immunoglobulin superfamily constant regions fused to the N-terminus of each of the V regions.

Also provided, in one embodiment, is one or more polynucleotides encoding the molecule of the present disclosure. In some embodiments, provided is a host cell comprising the one or more polynucleotides.

Further provided, in one embodiment, is a method for delivering an active antibody or antigen-binding fragment to a subject, comprising administering to the subject a molecule that comprises an immunoglobulin superfamily constant region and an antibody or antigen-binding fragment comprising a heavy chain variable region (VH), wherein the constant region is covalently coupled to the VH through a cleavable linker, wherein the cleavable linker is cleaved in the subject thereby releasing the antibody or antigen-binding fragment in the subject. In some embodiments, the method is for treating a disease or condition selected from the group consisting cancer, autoimmune disease, and infection.

### BRIEF DESCRIPTION OF THE DRAWINGS

FIG. 1 illustrates the structures of Formats 1-4.

FIG. 2 shows the results of human EGFR-His ELISA binding assays for Formats 1-4.

FIG. 3 shows the results of cell-based FACS binding assays for Formats 1-4.

FIG. 4 illustrates the structure of Formats 5-11.

FIG. 5 shows the results of cell-based FACS binding assays for Format 5 in comparison to Formats 1 and 2.

FIG. 6 shows the results of cell-based FACS binding assays for Formats 5-11 in comparison to Format 1.

FIG. 7 illustrates the structure of Formats 12-15.

FIG. 8 shows the results of cell-based FACS binding assays for Formats 12-14.

FIG. 9 shows the results of cell-based FACS binding, internalization and anti-mouse IgG MMAE mediated killing for Formats 15.

FIG. 10 illustrates the structure of Formats 18-22 and Format 25-27

FIG. 11 shows the results of cell-based FACS binding assays for Formats 18-22 and Formats 25-27.

FIG. 12 illustrates the structure of Formats 18-22 and Formats 28-32

FIG. 13 shows the results of cell-based FACS binding assays for Formats 28-30.

FIG. 14 shows the results of cell-based FACS binding and anti-mouse IgG MMAE mediated killing for Format 28 and activated Format 28

FIG. 15 compare the proteolysis efficacy of Formats 28 and Formats 30 by cell-based FACS binding and SDS-PAGE

FIG. 16 shows the results of ADC killing of Format 32-MMAE and activated Format 32-MMAE

FIG. 17 illustrates the structure of Formats 18-22 and Formats 33-34

FIG. 18 shows the results of cell-based FACS binding for Format 33 and Format 34

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)2, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG1 molecule and a hinge region derived from an IgG3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain constant region” includes amino acid sequences derived from antibody light chain. Preferably, the light chain constant region comprises at least one of a constant kappa domain or constant lambda domain.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

### Antibody Prodrugs

As discussed, a major challenge for developing an efficient and safe antibody prodrug platform is the identification of a suitable masking peptide. The masking peptide is ideally derived from a human protein to avoid immunogenicity in human subjects. More importantly perhaps, the masking peptide should have certain three-dimensional structure which effectively provides steric hindrance to the antibody. There is no clear understanding as to what kind of three-dimensional structure is required, however. If the structure requires a long sequence, however, the resulting prodrug may be too large, be difficult to manufacture, and be unstable. If the structure is too small, it may not be effective enough.

It is discovered, surprisingly, that the antibody CH3 domain can serve as an optimal masking peptide. All IgG, including IgG1, IgG2, IgG3 and IgG4, have highly homologous CH3 domains (see sequence alignment in Table A below).

TABLE A
Alignment of human IgG CH3 domains

| | |
|---|---|
| IgG1 | GQPREPQVYTLPPSREEMTKNQVSLTCLVKGFYPSDIAVEWESNGQPENNYKTTPPVLDS |
| IgG2 | GQPREPQVYTLPPSREEMTKNQVSLTCLVKGFYPSDIAVEWESNGQPENNYKTTPPMLDS |
| IgG3 | GQPREPQVYTLPPSREEMTKNQVSLTCLVKGFYPSDIAVEWESSGQPENNYNTTPPMLDS |
| IgG4 | GQPREPQVYTLPPSQEEMTKNQVSLTCLVKGFYPSDIAVEWESNGQPENNYKTTPPVLDS |
| IgG1 | DGSFFLYSKLTVDKSRWQQGNVFSCSVMHEALHNHYTQKSLSLSPGK (SEQ ID NO: 47) |
| IgG2 | DGSFFLYSKLTVDKSRWQQGNVFSCSVMHEALHNHYTQKSLSLSPGK (SEQ ID NO: 48) |
| IgG3 | DGSFFLYSKLTVDKSRWQQGNIFSCSVMHEALHNRFTQKSLSLSPGK (SEQ ID NO: 49) |
| IgG4 | DGSFFLYSRLTVDKSRWQEGNVFSCSVMHEALHNHYTQKSLSLSLGK (SEQ ID NO: 50) |

Small variations exist as well. For instance, E356 (EU numbering) may be D356, and M358 can be replaced by L358. An example variant is provided in SEQ ID NO: 10, with its secondary structural motifs annotated in Table B.

TABLE B
Secondary structure of CH3

```
CH3 (SEQ ID NO: 10)
GQPREPQVYTLPPSRDELTKNQVSLTCLVK [symbol] VEWESNGQPENN
              A-strand B-strand BC-loop C/D-strands
YKTTPPV [symbol] LYSKLTVDKSRWQQGNVFSC [symbol] QKS
                DE-turn E/F-strand FG-loop
LSLSPGK
G-strand
CH3 knob with S354C/T366W (SEQ ID NO: 19)
GQPREPQVYTLPPCRDELTKNQVSLWCLVK [symbol] VEWESNGQPENN
                                                              BC-loop
YKTTPPV [symbol] LYSKLTVDKSRWQQGNVFSC [symbol] QKS
                DE-turn FG-loop
LSLSPGK
CH3 hole with Y349C/T366S/L368A/Y407V (SEQ ID NO: 20)
GQPREPQVCTLPPSRDELTKNQVSLSCAVK [symbol] VEWESNGQPENN
                                                              BC-loop
YKTTPPV [symbol] LVSKLTVDKSRWQQGNVFSC [symbol] QKS
                DE-turn FG-loop
LSLSPGK
```

It is contemplated that the secondary motifs BC-loop (G371 through A378, EU numbering), DE-turn (L398 through F405, EU numbering), and FG-loop (S426 through T437, EU numbering), as well as the strands between them, form a suitable three-dimensional masking structure. As demonstrated in the experimental examples, the amino acid residues C-terminal to the FG-loop can be removed, and the resulting truncated CH3 domains exhibited even stronger masking effects. This portion of the CH3 domain, therefore, is referred to as the “loop-turn-loop” fragment hereinafter.

There are other immunoglobulin superfamily constant regions, such as IgG CH2, IgG CH1, IgG CL, and the T-cell receptor (TCR) constant region, which have similarly stable loop structures and no or low immunogenicity. For instance, in CH1, following an initial stretch (A114-K121), the A-strand (G122-S136) and B-strand (G137-K147), the BC-loop (D148-T155) is a stable loop structure (all according to EU number). Then, following the C-strand (V156-A162), CD-strand (L163-S165), and D-strand (G166-V173), there is a stable DE-turn (L174-S181); followed by E-strand (L182-L193), E-strand (G194-C200) and then the stable structure of FG-loop (N201-V211), which is followed by the G-strand (D212-V215, all according to EU numbering). Each of the BC-loop, DE-turn and FG-loop, and their combinations, serve to provide a strong masking effect. Residues in the initial stretch, the A-strand, B-strand and G-strand are contemplated to be removeable.

Likewise, in CH2, the secondary structures include an initial stretch (A231-G236), A-strand (G237-L251), AB-turn (M252-1253), B-strand (S254-V264), stable structure BC-loop (D265-K274), C-strand (F275-G281), CD-strand (V282-H285), D-strand (N286-E293), stable structure DE-turn (E294-R301), E-strand (V302-W313), F-strand (L314-C321), stable structure FG-loop (K322-1332), G-strand (E333-K340, all according to EU numbering). Each of the BC-loop, DE-turn and FG-loop, and their combinations, serve to provide a strong masking effect. Residues in the initial stretch, the A-strand, AB-turn, B-strand and G-strand are contemplated to be removeable.

Likewise, the present technology not only is applicable to full antibodies, but also to nanobodies and antigen-binding fragments, chimeric antigen receptors (CAR), and T-cell receptors (TCR). In some embodiments, the IgG CH3, IgG CH2, IgG CH1, IgG CL, and T-cell receptor (TCR) constant region are human constant regions.

In accordance with one embodiment of the present disclosure, therefore, provided is a molecule that includes an immunoglobulin superfamily constant region or a fragment thereof (e.g., for CH3, the fragment can be AB-turn, the DE-turn, the FG-loop, or a combination thereof) coupled, preferably covalently, to an immunoglobulin superfamily variable region. The variable region can be a heavy chain variable region (VH) or light chain variable region (VL) of an antibody or fragment, which encompasses both full-length conventional antibodies and single domain antibodies, as well as antigen-binding fragments. In some antibodies or antigen-binding fragments, such as a single-domain antibody (VHH), there is only a single variable region (e.g., VH). For such an antibody, a single constant region is needed. The immunoglobulin superfamily variable region, in another embodiment, is a TCR variable region.

In some embodiments, the molecule does not include an immunoglobulin superfamily variable fragment that is on the N-terminal side of the immunoglobulin superfamily constant region. In other words, the immunoglobulin superfamily constant region here is merely used as non-target-binding masking peptide.

In some embodiments, the covalent coupling of the immunoglobulin superfamily constant region to the immunoglobulin superfamily variable region inhibits the variable region's ability to bind to its binding target (e.g., antigen). In other words, after the immunoglobulin superfamily constant region is removed from the molecule, the remaining immunoglobulin superfamily variable region is able to bind its target molecule; before such removal, the whole molecule has reduced or no binding affinity to the target molecule. The immunoglobulin superfamily constant region, therefore, serves as a masking moiety.

More conventional antibodies have two or more variable regions. It is contemplated that only one immunoglobulin superfamily constant region is needed for each pair of VH/VL. This is because a VH/VL pair requires both variable regions to effectively bind an antigen. In some embodiments, the immunoglobulin superfamily constant region is coupled to the VH. In some embodiments, the immunoglobulin superfamily constant region is coupled to the VL. In a preferred embodiment, both VH and VL are coupled to immunoglobulin superfamily constant regions.

When both the VH and the VL are coupled to immunoglobulin superfamily constant regions, the two immunoglobulin superfamily constant regions can pair with each other which provides additional advantages of the present technology. On the one hand, the paired immunoglobulin superfamily constant regions form a larger and more stable steric structure that inhibits the binding activity of the VH/VL pair. On the other hand, when there are two or more pairs of constant regions in an antibody (e.g., bispecific or trispecific antibody), their pairing can be varied to reduce mispairing. In some embodiments, therefore, the two constant regions are modified, as compared to the wild-type constant regions, to increase the heterodimerization of the masking moiety.

For instance, in a conventional antibody that includes a pair of wild-type CH3 in the Fc region, two pairs of CH3 with knob-in-hole or charged-pair substitutions can be used as the masking moieties for both VH/VL pairs. In another example, in a bispecific antibody, one VH/VL pair can be fused to a pair of wildtype CH3 regions and the second VH/VL pair can be fused to a pair of CH3 regions with knob-in-hole or charged-pair substitutions, to reduce mispairing.

Besides CH3, CH1 and CL (lambda and kappa), and TCR alpha/beta chains, can also be paired, and can be mutated to form different pairings. Therefore, in one example, in a bispecific antibody, one VH/VL pair can be fused to a pair of wildtype CH1/CL regions and the second VH/VL pair can be fused to a pair of CH1/CL regions with knob-in-hole or charged-pair substitutions, to reduce mispairing.

In some embodiments, the pair of immunoglobulin superfamily constant regions is a pair of CH1 and CL, such as human IgG CH1 and CL. An example sequence of CH1 is provided as amino acid residues 1-98 in SEQ ID NO: 115, and an example sequence of CL is provided as SEQ ID NO: 7. In some embodiments, a few additional residues are inserted between the CH1 and the corresponding variable region (in addition to the optional linker therebetween). In other words, if counting such additional residues as a portion of the linker, then it means that the CH1 uses a longer linker than the CL to link to the corresponding variable regions.

In some embodiments, the additional residues are 1-10 residues, or 2-9, 2-8, 3-7, 4-6, or 5 amino acid residues. Such additional residues may be the whole or a fragment of a commonly used linker or hinge sequence. An example is EPKSC (SEQ ID NO: 120).

In some embodiments, the CH1 is fused, through the optional linker, to the VL in the VH/VL pair, and the CL is fused through the corresponding optional linker, to the VH in the VH/VL pair. In a less preferred embodiment, the CH1 is fused, through the optional linker, to the VH in the VH/VL pair, and the CL is fused through the corresponding optional linker, to the VL in the VH/VL pair. In some aspects of either embodiment, the CH1 connects to the corresponding variable region through a longer linker.

In some embodiments, the knob-in-hole substitutions include S354C and T366W in one of the CH3 domains, and Y349C, T366S, L368A, and Y407V in the other CH3 domain, according to EU numbering. In some embodiments, the charge-pair substitutions include K409D/D399R, K409E/D399K, or K409E/D399R.

In some embodiments, the pairing between the CH3 regions, the CH1 and CL, or the TCR alpha/beta chains, of their fragments, can be further enhanced. For instance, a disulfide bond can be generated between the paired constant regions when a suitable cysteine is introduced each sequence. Other than disulfide bonds, chemical linkers can also be used, without limitation. It is contemplated, when the enhanced pairing is used, the stronger pairing allows the use of even short fragments of the constant regions (as exemplified herein) to serve as effective masking moieties.

In some embodiments, only a single pair of such constant regions is included in the molecule. As shown in the experimental examples, a single pair (CH3/CH3) is sufficient to inhibit the antibody activities, and thus adding a second pair (e.g., CH2-CH3/CH2-CH3) is not required. In some embodiments, at the N-terminal side of the variable regions of the binding unit (such as the VH/VL), there are no other functional unit except the single pair of immunoglobulin superfamily constant regions. A “functional unit,” as used herein, refers to protein domains involved in antibody binding, stabilization, or circulation. An exception to a functional unit is a signal peptide.

In some embodiments, the peptide portion at the N-terminal side of the variable regions of the binding unit (such as the VH/VL) is not longer than 200 amino acid residues (not counting an optional signal peptide). In some embodiments, this N-terminal portion is not longer than 190, 180, 170, 160, 150, 140, 130, 120, 110, or 105 amino acid residues (not counting an optional signal peptide).

Also provided, in one embodiment, is a fusion protein that includes a peptide linker fused to the C-terminus of an immunoglobulin superfamily constant region. The peptide linker, in some embodiments, can be further fused to the N-terminus of an immunoglobulin superfamily variable region as needed, as described above. In some embodiments, the fusion protein does not include an immunoglobulin superfamily variable fragment that is on the N-terminal side of the immunoglobulin superfamily constant region. In other words, the immunoglobulin superfamily constant region here is merely used as non-target-binding masking peptide.

In some embodiments, the fusion protein is provided as a pair, such as a pair of CH3, a CH1 and a CL, or a TCR alpha chain and a TCR beta chain, each is fused to a peptide linker. In some embodiments, the pair is modified to include knob-in-hole or charge-pair pairing. In some embodiments, the pairing between the CH3 regions, the CH1 and CL, or the TCR alpha/beta chains, of their fragments, can be further enhanced. For instance, a disulfide bond can be generated between the paired constant regions when a suitable cysteine is introduced each sequence. Other than disulfide bonds, chemical linkers can also be used, without limitation. It is contemplated, when the enhanced pairing is used, the stronger pairing allows the use of even short fragments of the constant regions to serve as effective masking moieties.

In some embodiments, there are no more than 50, 45, 40, 35, 30, 25, 24, 23, 22, 21, 20, 19, 18, 17, 16, or 15 amino acid residues, or preferably no more than 14, 13, 12, 11, 10, 9, or 8 amino acid residues, between T437 of CH3 according to EU numbering (T468 according to Kabat numbering) (or V211 (EU numbering) of CH1, or 1332 (EU numbering) of CH2), of the variable region and the C-terminus of the cleavable peptide linker.
there are at least 8 amino acid residues, preferably at least 9, 10, 11 or 12 amino acid residues, and more preferably at least 13, 14, 15 16, 17, 18, 19 or 20 amino acid residues between T437, according to EU numbering (T468 according to Kabat numbering), of each CH3 domain and the N-terminus of the corresponding variable region.

In some embodiments, there are 8 to 23 amino acid residues between T437, according to EU numbering (T468 according to Kabat numbering), of each CH3 domain and the N-terminus of the corresponding variable region.

In some embodiments, there are 12 to 20 amino acid residues between T437, according to EU numbering (T468 according to Kabat numbering), of each CH3 domain and the N-terminus of the corresponding variable region.

In some embodiments, the constant region is truncated to at least retain a fragment which is sufficient to inhibit the binding of the variable region to the target molecule. In some embodiments, the CH3 domain is truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, C-terminal amino acid residue(s) as compared to the wild-type human IgG CH3 domain. In some embodiments, the CH3 domain is truncated to remove at least one, preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, C-terminal amino acid residue as compared to the wild-type variable region. Examples of immunoglobulin superfamily constant region and peptide linkers are described in further detail throughout the disclosure.

Also provided, in some embodiments, is a molecule that includes an immunoglobulin superfamily constant region coupled, preferably covalently, to a T-cell receptor (TCR). In some embodiments, the immunoglobulin superfamily constant region is coupled to a variable (V) region of the TCR. In some embodiments, an immunoglobulin superfamily constant region is coupled to each variable (V) region of the TCR.

As demonstrated, the immunoglobulin superfamily constant region here is sufficient to effectively block or reduce the activity of the antibody, fragment or T-cell receptor. In some embodiments, therefore, the molecule does not include further domains in the masking moiety. In some embodiments, there is no variable region (VH, VL, or TCR variable region etc.) together with the immunoglobulin superfamily constant region. In some embodiments, there is no variable region (VH, VL, or TCR variable region etc.) that is disposed N-terminal to the immunoglobulin superfamily constant region. In some embodiments, there is no variable region (VH, VL, or TCR variable region etc.) that is disposed between the immunoglobulin superfamily constant region and the antibody, antigen-binding fragment or TCR. In some embodiment, the masking moiety includes a single constant region (e.g., a single CH3 without CH1 or CH2).

In some embodiments, the variable region(s) for an antigen-binding unit, such as a full-sized Fab antibody, a nanobody, a single-chain fragment, or a Bispecific T cell engager (BiTE). In some embodiments, the antigen-binding unit includes a VH and VL pair, or a pair of nanobodies.

As an antibody prodrug, the masking peptide should stay in the prodrug in non-target tissues and be removed at the target tissue. The removal, in some embodiments, can be achieved by removal, degradation, breakage, or digestion of a linker that couples the masking peptide to the antibody, antigen-binding fragment or TCR. An example is an enzymatically cleavable peptide linker.

In some embodiment, the enzyme (protease) that can cleave the peptide linker is uniquely expressed or overexpressed at a diseased tissue or organ, compared to healthy tissue or organ. Preferably, the enzyme is found in the extracellular environment of the diseased tissue or organ. Examples of such proteases include: aspartate proteases (e.g., renin), fibroblast activation protein (FAP), aspartic cathepsins (e.g., cathepsin D, caspase 1, caspase 2, etc.), cysteine cathepsins (e.g., cathepsin B), cysteine proteases (e.g., legumain), disintegrin/metalloproteinases (ADAMs, e.g., ADAM8, ADAM9), disintegrin/metalloproteinases with thrombospondin motifs (ADAMTS, e.g., ADAMTS1), integral membrane serine proteases (e.g., matriptase 2, MT-SPI/matriptase, TMPRSS2, TMPRSS3, TMPRSS4), kallikrein-related peptidases (KLKs, e.g. KLK4, KLK5), matrix metalloproteases (e.g., MMP-1, MMP-2, MMP-9), and serine proteases (e.g., cathepsin A, coagulation factor proteases such as elastase, plasmin, thrombin, PSA, uPA, Factor Vila, Factor Xa, and HCV NS3/4). Preferably, the protease is fibroblast activation protein (FAP), urokinase-type plasminogen activator (uPA, urokinase), MT-SPI/matriptase, legumain, or a matrix metalloprotease (especially MMP-1, MMP-2, and MMP-9). Those skilled in the art will appreciate that the choice of the enzyme and the corresponding cleavable peptide will depend on the disease to be treated and the protease(s) expressed by the affected tissue or organ.

Example enzymatically cleavable peptide linkers are provided in Table C.

TABLE C
Example enzymatically cleavable peptide linkers

| Cleavable Linker | SEQ ID NO: | Cleaving enzyme |
|---|---|---|
| LSGRSDNH | 51 | Urokinase, matriptase, legumain |
| LSGK | 52 | Matriptase |
| LSGX (X is R or K) | 53 | Matriptase |
| VPLSLYS | 54 | Matrix metalloprotease-9 (MMP-9) |
| PLGLAG | 55 | Matrix metalloprotease-2 (MMP-2) |
| IPVSLRSG | 64 | Matrix metalloprotease-2 (MMP-2) |
| VLVPMAMMAS | 56 | Matrix metalloprotease-1 (MMP-1) |
| XXQAR(A/V)X (X is any | 57 | Matriptase (MT-SP1) |
| amino acid) | | |
| AGPR | 58 | Matriptase (MT-SP1) |
| AANL | 59 | Legumain |
| PTNL | 60 | Legumain |
| TSGRSANP | 61 | Various |
| DEXXXC(A/S) (X is any | 62 | HCV NS3/4 |
| amino acid) | | |
| DLXXXT(A/S) (X is any | 63 | HCV NS3/4 |
| amino acid) | | |
| PLGLAGGGS | 101 | MMP-2 |
| PLGLAGGGGS | 102 | MMP-2 |
| IPVSLRSGPLGLAG | 103 | MMP-2 |

In some embodiments, each peptide linker in each of the one or more protein chains is capable of being cleaved by the same cleaving enzyme, such that once the enzyme is present, all of the linkers will be cleaved at the same time, fully activating the antibody. In some embodiments, each peptide linker has the same sequence.

In some embodiments, the peptide linker includes a sequence selected from SEQ ID NO: 51-63 or 101-103. In some embodiments, the peptide linker includes two enzymatic cleavage sites, such as SEQ ID NO: 103. In some embodiments, the peptide linker includes additional amino acid residues such as G (glycine) and S (serine).

The accompanying experimental examples have demonstrated that when some of the C-terminal amino acid residues of the full-length CH3 domain were removed, the resulting CH3 fragments exhibited even stronger masking effects than their full-length counterparts. It is contemplated that this is because the loop-turn-loop fragment of the CH3 domain, with the truncation, is spatially closer to the variable regions. Such closer spatial relationship, it is contemplated, leads to higher steric hinderance.

The term “CH3 domain” as used in the present disclosure, encompasses both sequence homologues of the wild-type CH3 domains as well as their fragments that include at least the loop-turn-loop portion.

Sequences of wild-type human IgG CH3 domains are provided in SEQ ID NO: 47-50 (Table A). Their sequence homologues include those with conservative amino acid substitutions (e.g., SEQ ID NO: 10) and those with knob-in-hole modifications (e.g., SEQ ID NO: 19-20).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE D
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE E
Conservative Amino Acid Substitutions

| For Amino | |
|---|---|
| Acid | Substitution With |
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, |
| | L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

For each sequence homologue of a full-length CH3 domain, its fragments are also within the meaning of a CH3 domain, so long as the fragment includes at least the loop-turn-loop portion. As provided, the loop-turn-loop fragment of a full-length CH3 domain includes the BC-loop (G371 through A378, EU numbering), DE-turn (L398 through F405, EU numbering), and FG-loop (S426 through T437, EU numbering), as well as the strands (e.g., C-strand, CD-strand, D-strand, E-strand, and F-strand) between them. The A-strand, B-strand and G-strand are not within this loop-turn-loop fragment, and thus can be removed, partially or completely.

In some embodiments, the CH3 domain has a truncation but it at least retains a fragment which is sufficient to inhibit the binding of the variable region to the target molecule. In some embodiments, the truncation is at the C-terminal end. In some embodiments, the last amino acid (K447, EU numbering) with reference to SEQ ID NO: 10 is removed. In some embodiments, the last two amino acids (G446-K447, EU numbering) with reference to SEQ ID NO: 10 are removed. In some embodiments, the last three amino acids (P445-G446-K447, EU numbering) with reference to SEQ ID NO: 10 are removed. In some embodiments, the last four amino acids (S444-P445-G446-K447, EU numbering) with reference to SEQ ID NO: 10 are removed. In some embodiments, the last five amino acids (L443-S444-P445-G446-K447, EU numbering) with reference to SEQ ID NO: 10 are removed. In some embodiments, the last six amino acids (S442-L443-S444-P445-G446-K447, EU numbering) with reference to SEQ ID NO: 10 are removed. In some embodiments, the last seven amino acids (L441-S442-L443-S444-P445-G446-K447, EU numbering) with reference to SEQ ID NO: 10 are removed. In some embodiments, the last eight amino acids (S440-L441-S442-L443-S444-P445-G446-K447, EU numbering) with reference to SEQ ID NO: 10 are removed. In some embodiments, the last eight amino acids (S440-L441-S442-L443-S444-P445-G446-K447, EU numbering) with reference to SEQ ID NO: 10 are removed. In some embodiments, the last nine amino acids (K439-S440-L441-S442-L443-S444-P445-G446-K447, EU numbering) with reference to SEQ ID NO: 10 are removed. In some embodiments, the last ten amino acids (Q438-K439-S440-L441-S442-L443-S444-P445-G446-K447, EU numbering) with reference to SEQ ID NO: 10 are removed.

In some embodiments, the CH3 domain has a truncation but it at least retains a fragment which is sufficient to inhibit the binding of the variable region to the target molecule. In some embodiments, the CH3 domain is truncated at the N-terminus, so long as the BC-loop (G371 through A378, EU numbering) is kept intact. In some embodiments, the CH3 domain is truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 N-terminal amino acid residue(s) as compared to the wild-type human IgG CH3 domain. In some embodiments, the CH3 domain includes amino acid residues G371 through T437 of a full-length CH3 domain. In some embodiments, the CH3 domain includes amino acid residues K360 through T437 of a full-length CH3 domain. In some embodiments, the CH3 domain includes amino acid residues E345 through T437 of a full-length CH3 domain.

In some embodiments, the CH3 domain includes amino acid residues 31-97, 20-97, 10-97, 5-97, 4-97, 3-97, 2-97, or 5-101 of SEQ ID NO: 10, 19, 20, 47, 48, 49 or 50. In some embodiments, the CH3 domain includes amino acid residues 1-97 of SEQ ID NO: 10, 19, 20, 47, 48, 49 or 50. In some embodiments, one of the CH3 domain (e.g., the one fused to VL) includes amino acid residues 1-97 of SEQ ID NO: 19 and the other CH3 domain (e.g., the one fused to VH) includes amino acid residues 1-97 of SEQ ID NO: 20.

Likewise, when CL, CH1 or CH2 is used, the CL, CH1 or CH2 can also be truncated at the N-terminus of the C-terminus. In some embodiment, the CH1 domain is truncated to remove at least one, or preferably at least 2, 3, 4, 5, 6, 7, 8, 9 or 10, C-terminal amino acid residue(s) as compared to the wild-type human IgG CH1 domain. In some embodiments, the CH1 domain is truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, N-terminal amino acid residue(s) as compared to the wild-type human IgG CH1 domain.

In some embodiment, the CL domain is truncated to remove at least one, or preferably at least 2, 3, 4, 5, 6, 7, 8, 9 or 10, C-terminal amino acid residue(s) as compared to the wild-type human IgG CL domain. In some embodiments, the CL domain is truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, N-terminal amino acid residue(s) as compared to the wild-type human IgG CL domain.

In some embodiment, the CH2 domain is truncated to remove at least one, or preferably at least 2, 3, 4, 5, 6, 7, 8, 9 or 10, C-terminal amino acid residue(s) as compared to the wild-type human IgG CH2 domain. In some embodiments, the CH2 domain is truncated to remove at least one, or preferably at least 2, 3, 4, 5, 6, 7, 8, 9, 10, 11, 12, 13, 14, 15, 16, 17, 18, 19, 20, 21, 22, 23, 24, 25, 26, 27, 28, 29 or 30, N-terminal amino acid residue(s) as compared to the wild-type human IgG CH2 domain.

In some embodiments, the distance between the C-terminus of the FG-loop (i.e., T437 of CH3, according to EU numbering, or T468 according to Kabat numbering) is limited to ensure sufficient steric hinderance. In some embodiments, there are no more than 50, 45, 40, 35, 30, 25, 20, or 15 amino acid residues between CH3 T437 (EU numbering) and the N-terminus of the corresponding variable region. In some embodiments, there are no more than 14 amino acid residues between CH3 T437 (EU numbering) and the N-terminus of the corresponding variable region. In some embodiments, there are no more than 13, 12, 11, 10, 9, 8, 7, 6 or 5 amino acid residues between CH3 T437 (EU numbering) and the N-terminus of the corresponding variable region.

Likewise, in some embodiments, there are no more than 50, 45, 40, 35, 30, 25, 20, 15 or 14 amino acid residues between V211 (EU numbering) of CH1 and the N-terminus of the corresponding variable region. In some embodiments, there are no more than 13, 12, 11, 10, 9, 8, 7, 6 or 5 amino acid residues between V211 (EU numbering) of CH1 and the N-terminus of the corresponding variable region.

In some embodiments, there are no more than 50, 45, 40, 35, 30, 25, 20, 15 or 14 amino acid residues between 1332 (EU numbering) of CH2 and the N-terminus of the corresponding variable region. In some embodiments, there are no more than 13, 12, 11, 10, 9, 8, 7, 6 or 5 amino acid residues between 1332 (EU numbering) of CH2 and the N-terminus of the corresponding variable region.

In some embodiments, the immunoglobulin superfamily constant region, such as CH3, is conjugated to the antibody, fragment, or TCR through a chemical linker. In some embodiment, the chemical linker is covalently attached to an amino acid of a variable region. In some embodiments, the amino acid is in the framework region. In some embodiment, the amino acid is a framework region N-terminal to all CDRs.

In some embodiments, the chemical linker is a cleavable linker. The cleavable linker may be cleaved by proteolytic enzymes or can be acidically activated in a microenvironment of a disease. In some embodiments, the linker is covalently linked to an amino acid in the antibody, such as cysteine. In some embodiments, the cleavable linker is a peptide capable of being cleaved by one or multiple proteolytic enzyme, protease or peptidase, wherein the protease is selected from the group consisting of cysteine protease, asparagine protease, aspartate protease, glutamic acid protease, threonine protease, gelatinase, metalloproteinase, or asparagine peptide lyase, or is a bond cleavable in an acidic condition of a pathologic microenvironment. In some embodiments, the cleavable linker is selected from the group consisting of amide, ester, carbamate, urea and hydrazone bonds.

The antibody or fragment included in the fusion molecule can have specificity to any antigen and have any antibody or fragment structure. In some embodiments, it has a conventional Fab structure with a Fc fragment. In some embodiments, it includes at least a VH/VL pair. In some embodiments, it has a single variable region. In some embodiments, the antibody or fragment has specificity to a tumor antigen.

A “tumor antigen” is an antigenic substance produced in tumor cells, i.e., it triggers an immune response in the host. Tumor antigens are useful in identifying tumor cells and are potential candidates for use in cancer therapy. Normal proteins in the body are not antigenic. Certain proteins, however, are produced or overexpressed during tumorigenesis and thus appear “foreign” to the body. This may include normal proteins that are well sequestered from the immune system, proteins that are normally produced in extremely small quantities, proteins that are normally produced only in certain stages of development, or proteins whose structure is modified due to mutation.

An abundance of tumor antigens are known in the art and new tumor antigens can be readily identified by screening. Non-limiting examples of tumor antigens include EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGFIR, EPHA3, TRAILR1, TRAILR2, RANKL, FAP and Tenascin.

In some embodiments, the antibody or antigen binding fragment binds an antigen expressed on the surface of an immune cell. In some embodiments, the antibody or antigen binding fragment binds to a cluster of differentiation molecule selected from the group consisting of: CD1a, CD1b, CD1c, CD1d, CD2, CD3, CD4, CD5, CD6, CD7, CDS, CD9, CD 10, CD11A, CD11B, CD 11C, CDwl 2, CD13, CD14, CD15, CD15s, CD16, CDwl7, CD18, CD19, CD20, CD21, CD22, CD23, CD24, CD25, CD26, CD27, CD28, CD29, CD3Q, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD6I, CD62E, CD62L, CD62P, CD63, CD64, CD65, CD66a, CD66b, CD66c, CD66d, CD66e, CD66E CD68, CD69, CD70, CD71, CD72, CD73, CD74, CD75, CD76, CD79o, 0O79b, CD80, CD81, CD82, CD83, CDw84, CD85, CD86, CD87, CD88, CD89, CD90, CD91, CDw92, CD93, CD94, CD95, CD96, CD97, CD98, CD99, CD 100, CDIGI, CD 102, CD103, CD104, CD105, CD106, CD107a, CD107b, CDw′108, CD109, CD114, CD115, CD116. CD117. CD118, CD119, CD120a, CD120b, CD121a, CDwl21b, CD122, CD123, CD124, CD125, CD126, CD127, CDwl28, CD129, CD130, CDwl31, CD132, CD134, CD135, CDwl36, CDwl37, CD138, CD139, CD140a, CD140b, CD141, CD142, CD143, CD144, CD145, CD146, CD147, CD148, CD15G, CD151, CD152, CD153, CD 154, CD155, CD156, CD157, CD158a, CD158b, CD161, CD162, CD163, CD164, CD165, CD166, and CD182.

In some embodiments, the antibody or antigen binding fragment binds an antigen selected from the group consisting of a hormone, growth factor, cytokine, a cell-surface receptor, or any ligand thereof. In some embodiments, the antibody or antigen binding fragment binds an antigen selected from the group consisting of such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, If-15. IL-16, IL-17, IL-18, IFN, TNF a, TNF1, TNF2, G-CSF, Meg-CSF, GM-CSF, thrombopoietin, stem cell factor, and erythropoietin. In some embodiments, the antibody is Cetuximab, which has a VH of SEQ ID NO: 1 and a VL of SEQ ID NO: 6. In some embodiment, the antibody has a heavy chain of SEQ ID NO: 8 and a light chain of SEQ ID NO: 9.

In some embodiments, the antibody prodrug includes a heavy chain having the amino sequence of SEQ ID NO: 11 and a light chain having the amino sequence of SEQ ID NO: 12. In some embodiments, the antibody prodrug includes a heavy chain having the amino sequence of SEQ ID NO: 13 and a light chain having the amino sequence of SEQ ID NO: 14. In some embodiments, the antibody prodrug includes a heavy chain having the amino sequence of SEQ ID NO: 15 and a light chain having the amino sequence of SEQ ID NO: 16.

In some embodiments, the antibody prodrug includes a heavy chain having the amino sequence of SEQ ID NO: 21 and a light chain having the amino sequence of SEQ ID NO: 22. In some embodiments, the antibody prodrug includes a heavy chain having the amino sequence of SEQ ID NO: 23 and a light chain having the amino sequence of SEQ ID NO: 24. In some embodiments, the antibody prodrug includes a heavy chain having the amino sequence of SEQ ID NO: 25 and a light chain having the amino sequence of SEQ ID NO: 26. In some embodiments, the antibody prodrug includes a heavy chain having the amino sequence of SEQ ID NO: 27 and a light chain having the amino sequence of SEQ ID NO: 28. In some embodiments, the antibody prodrug includes a heavy chain having the amino sequence of SEQ ID NO: 29 and a light chain having the amino sequence of SEQ ID NO: 30. In some embodiments, the antibody prodrug includes a heavy chain having the amino sequence of SEQ ID NO: 31 and a light chain having the amino sequence of SEQ ID NO: 32. In some embodiments, the antibody prodrug includes a heavy chain having the amino sequence of SEQ ID NO: 33 and a light chain having the amino sequence of SEQ ID NO: 34.

Methods of using the disclosed molecules are also provided. In one embodiment, provided is a method for delivering an active antibody or antigen-binding fragment, or a TCR, to a subject, such as a human subject. In some embodiments, the method entails administering to the subject a molecule of the present disclosure, wherein the cleavable linker is cleaved in the subject thereby releasing the antibody or antigen-binding fragment, or TCR, in the subject.

The methods may be useful for treating a disease or condition, such as cancer, autoimmune disease, and infection.

### Polynucleotides Encoding the Polypeptides and Methods of Preparing the Polypeptides

The present disclosure also provides isolated polynucleotides or nucleic acid molecules (such as DNA and mRNA, without limitation) encoding the fusion molecules, variants or derivatives thereof of the disclosure. Also provided are vectors, constructs, and cells that include the polynucleotides or nucleic acid molecules. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making proteins and antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Compositions

The present disclosure also provides pharmaceutical compositions. Such compositions comprise an effective amount of a fusion molecule, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1: Antibody Prodrugs with Masking CH3 Domains

This example prepared a series of prodrugs based on Cetuximab that included a pair of human IgG1 CH3 fragments as the masking moiety. Each prodrug included linkers of different lengths.

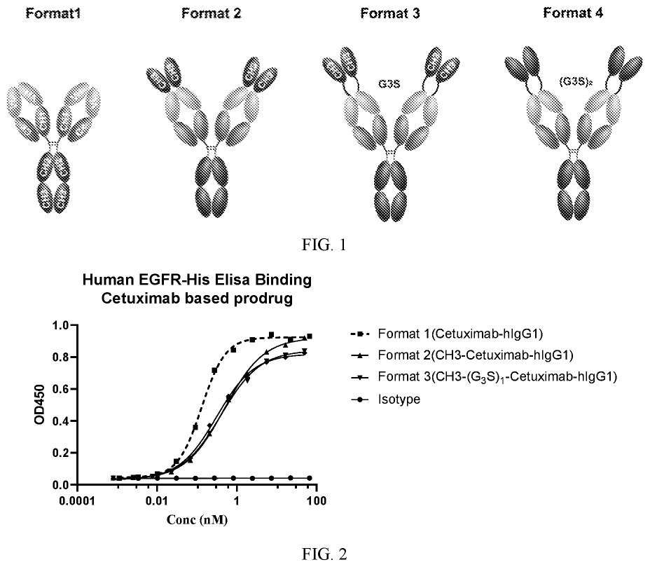

The tested prodrugs are listed in Table 1, illustrated in FIG. 1 and with sequences provided in Table 2. Format 1 is the parental antibody Cetuximab. Format 2 adds a pair of wild-type CH3 domains to the N-terminus of the VH and VL of the parental antibody. In Format 3 and Format 4, a peptide linker (GGGS (SEQ ID NO: 17) or GGGSGGGS (SEQ ID NO: 18)) was inserted between the CH3 domain and the parental antibody.

TABLE 1
Tested Antibody Prodrugs

| Format # | Heavy Chain | Light Chain |
|---|---|---|
| 1 | VH + CH1 + Hinge + CH2 + CH3 | VL + CLκ |
| 2 | CH3 + VH + CH1 + Hinge + CH2 + CH3 | CH3 + VL + CLκ |
| 3 | CH3 + (G₃S)₁ + VH + CH1 + | CH3 + (G₃S)₁ + |
| | Hinge + CH2 + CH3 | VL + CLκ |
| 4 | CH3 + (G₃S)₂ + VH + CH1 + | CH3 + (G₃S)₂ + |
| | Hinge + CH2 + CH3 | VL + CLκ |

TABLE 2
Protein Sequences of Formats 1-4

| | | SEQ ID |
|---|---|---|
| Name | Sequence | NO: |
| Parental antibody | Cetuximab VH (SEQ ID NO: 1) | 8 |
| (Format 1) heavy | QVQLKQSGPGLVQPSQSLSITCTVSGFSLTNYGVHWVRQSPGKG | |
| chain | LEWLGVIWSGGNTDYNTPFTSRLSINKDNSKSQVFFKMNSLQSN | |
| | DTAIYYCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSN | |
| | TKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKF | |
| | NWYVDGVEVHNAKTKPREEQYNSTYRVVSVLTVLHQDWLNGK | |
| | EYKCKVSNKALPAPIEKTISKAK | |
| | CH3 (SEQ ID NO: 5) | |
| | GQPREPQVYTLPPSREEMTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| Parental antibody | Cetuximab VL (SEQ ID NO: 6) | 9 |
| (Format 1) light | DILLTQSPVILSVSPGERVSFSCRASQSIGTNIHWYQQRTNGSPRLL | |
| chain | IKYASESISGIPSRFSGSGSGTDFTLSINSVESEDIADYYCQQNNNW | |
| | PTTFGAGTKLELK | |
| | CLκ (SEQ ID NO: 7) | |
| | RTVAAPSVFIFPPSDEQLKSGTASVVCLLNNFYPREAKVQWKVD | |
| | NALQSGNSQESVTEQDSKDSTYSLSSTLTLSKADYEKHKVYACE | |
| | VTHQGLSSPVTKSFNRGEC | |
| CH3 wild-type | GQPREPQVYTLPPSRDELTKNQVSLTCLVKGFYPSDIAVEWESNG | 10 |
| (WT) | QPENNYKTTPPVLDSDGSFFLYSKLTVDKSRWQQGNVFSCSVMH | |
| | EALHNHYTQKSLSLSPGK | |
| Format 2 heavy | CH3 WT (SEQ ID NO: 10) | 11 |
| chain | GQPREPQVYTLPPSRDELTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| | Cetuximab VH (SEQ ID NO: 1) | |
| | QVQLKQSGPGLVQPSQSLSITCTVSGFSLTNYGVHWVRQSPGKG | |
| | LEWLGVIWSGGNTDYNTPFTSRLSINKDNSKSQVFFKMNSLQSN | |
| | DTAIYYCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSN | |
| | TKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKF | |
| | NWYVDGVEVHNAKTKPREEQYNSTYRVVSVLTVLHQDWLNGK | |
| | EYKCKVSNKALPAPIEKTISKAK | |
| | CH3 (SEQ ID NO: 5) | |
| | GQPREPQVYTLPPSREEMTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| Format 2 light | CH3 WT (SEQ ID NO: 10) | 12 |
| chain | GQPREPQVYTLPPSRDELTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| | Cetuximab VL (SEQ ID NO: 6) | |
| | DILLTQSPVILSVSPGER VSFSCRASQSIGTNIHWYQQRTNGSPRLL | |
| | IKYASESISGIPSRFSGSGSGTDFTLSINSVESEDIADYYCQQNNNW | |
| | PTTFGAGTKLELK | |
| | CLκ (SEQ ID NO: 7) | |
| | RTVAAPSVFIFPPSDEQLKSGTASVVCLLNNFYPREAKVQWKVD | |
| | NALQSGNSQESVTEQDSKDSTYSLSSTLTLSKADYEKHKVYACE | |
| | VTHQGLSSPVTKSFNRGEC | |
| Format 3 heavy | CH3 WT (SEQ ID NO: 10) | 13 |
| chain | GQPREPQVYTLPPSRDELTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKG | |
| | LEWLGVIWSGGNTDYNTPFTSRLSINKDNSKSQVFFKMNSLQSN | |
| | DTAIYYCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSN | |
| | TKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKF | |
| | NWYVDGVEVHNAKTKPREEQYNSTYRVVSVLTVLHQDWLNGK | |
| | EYKCKVSNKALPAPIEKTISKAK | |
| | CH3 (SEQ ID NO: 5) | |
| | GQPREPQVYTLPPSREEMTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| Format 3 light | CH3 WT (SEQ ID NO: 10) | 14 |
| chain | GQPREPQVYTLPPSRDELTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRINGSPRLL | |
| | IKYASESISGIPSRFSGSGSGTDFTLSINSVESEDIADYYCQQNNNW | |
| | PTTFGAGTKLELK | |
| | CLκ (SEQ ID NO: 7) | |
| | RTVAAPSVFIFPPSDEQLKSGTASVVCLLNNFYPREAKVQWKVD | |
| | NALQSGNSQESVTEQDSKDSTYSLSSTLTLSKADYEKHKVYACE | |
| | VTHQGLSSPVTKSFNRGEC | |
| Format 4 heavy | CH3 WT (SEQ ID NO: 10) | 15 |
| chain | GQPREPQVYTLPPSRDELTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| | Linker (SEQ ID NO: 18) | |
| | GGGSGGGS | |
| | Cetuximab VH (SEQ ID NO: 1) | |
| | QVQLKQSGPGLVQPSQSLSITCTVSGFSLTNYGVHWVRQSPGKG | |
| | LEWLGVIWSGGNTDYNTPFTSRLSINKDNSKSQVFFKMNSLQSN | |
| | DTAIYYCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGA | |
| | LTSGVHTFPAVLQSSGLYSLSSVVTVPSSSLGTQTYICNVNHKPSN | |
| | TKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKF | |
| | NWYVDGVEVHNAKTKPREEQYNSTYRVVSVLTVLHQDWLNGK | |
| | EYKCKVSNKA | |
| | LPAPIEKTISKAK | |
| | CH3 (SEQ ID NO: 5) | |
| | GQPREPQVYTLPPSREEMTKNQVSLTCLVKGFYPSDIAVEWESNG | |
| | QPENNYKTTPPVLDSDGSFFLYSKLTVDKSRWQQGNVFSCSVMH | |
| | EALHNHYTQKS | |
| | LSLSPGK | |
| Format 4 light | CH3 WT (SEQ ID NO: 10) | 16 |
| chain | GQPREPQVYTLPPSRDELTKNQVSLTCLVKGFYPSDIAVEWESNG | |
| | QPENNYKTTPPVLDSDGSFFLYSKLTVDKSRWQQGNVFSCSVMH | |
| | EALHNHYTQKSLSLSPGK | |
| | Linker (SEQ ID NO: 18) | |
| | GGGSGGGS | |
| | Cetuximab VL (SEQ ID NO: 6) | |
| | DILLTQSPVILSVSPGERVSFSCRASQSIGTNIHWYQQRTNGSPRLL | |
| | IKYASESISGIPSRFSGSGSGTDFTLSINSVESEDIADYYCQQNNNW | |
| | PTTFGAGTKLELK | |
| | CLκ (SEQ ID NO: 7) | |
| | RTVAAPSVFIFPPSDEQLKSGTASVVCLLNNFYPREAKVQWKVD | |
| | NALQSGNSQESVTEQDSKDSTYSLSSTLTLSKADYEKHKVYACE | |
| | VTHQGLSSPVTKSFNRGEC | |

These antibody prodrugs were tested for their binding to human EGFR using ELISA. The results are presented in FIG. 2 and summarized in Table 3.

TABLE 3
ELISA Results

| Molecule | EC50 (μg/mL) |
|---|---|
| Isotype (IgG) | — |
| Format 1 | 0.01968 |
| Format 2 | 0.09827 |
| Format 3 | 0.08061 |
| Format 4 | 0.06207 |

The results show that Format 2 had an about 5-fold decrease of affinity to EGFR compared to Format 1, demonstrating the validity of the CH3 domains as a masking moiety.

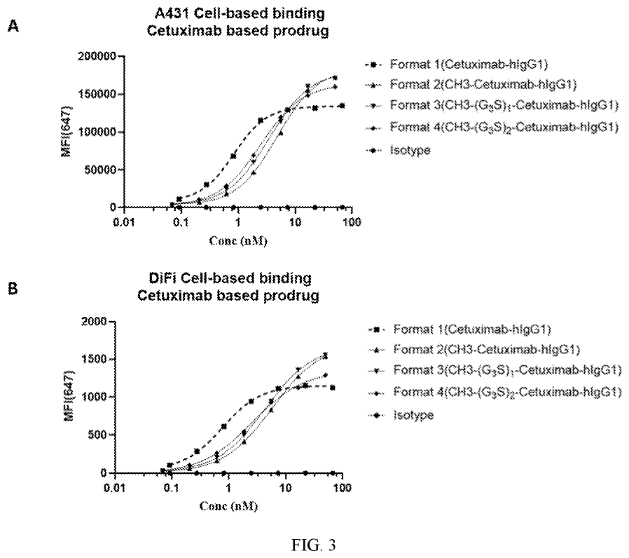

These molecules were also tested with two EGFR expressing tumor cell-based FACS binding assays (with A431 cells and DiFi cells). As shown in FIG. 3 and Tables 4-5, Format 2 also exhibited the highest inhibition (8˜10 fold) on the antibody affinity.

TABLE 4
Cell-based FACS Results (A431 cells)

| Molecule | EC50 (μg/mL) |
|---|---|
| Isotype (IgG) | — |
| Format 1 | 0.1260 |
| Format 2 | 0.9530 |
| Format 3 | 0.6937 |
| Format 4 | 0.4895 |

TABLE 5
Cell-based FACS Results (DiFi cells)

| Molecule | EC50 (μg/mL) |
|---|---|
| Isotype (IgG) | — |
| Format 1 | 0.1175 |
| Format 2 | 1.170 |
| Format 3 | 0.8716 |
| Format 4 | 0.5320 |

Formats 3 and 4, which included linkers between the masking domains and the variable domains, exhibited less reduction of activity in both experiments. These data, therefore, suggest that longer distances may reduce the masking effect of the CH3 domains.

### Example 2: C-Terminally Truncated CH3 Domains with Knob-into-Hole Mutations as Masking Moiety

Based on the results of Example 1, this example designed antibody prodrugs with CH3 domains with two types of modifications. One is that the pair of CH3 domains incorporated knob-in-hole mutations (e.g., as shown in SEQ ID NO: 19-20), and the other is C-terminal truncations at different lengths.

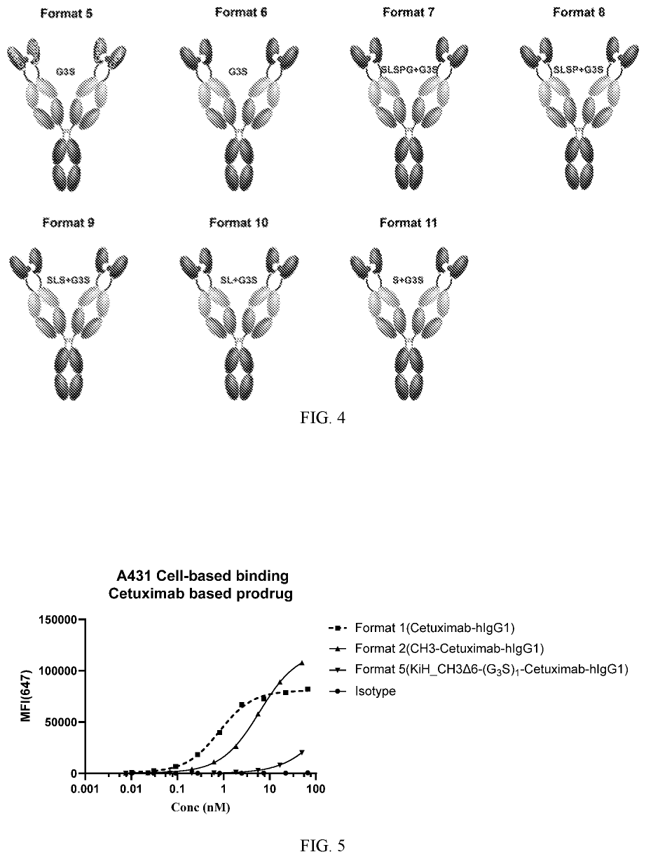

These new antibody prodrugs, referred to as Formats 5-11, are described in Table 6, illustrated in FIG. 4, and with sequences shown in Table 7.

In Format 5, the CH3 domains included both types of modifications, i.e., knob-in-hole and six amino acids truncation (46) at the C-terminus. More specifically, the “CH3 hole” was fused to the N-terminus of the VH of the parental antibody, and the “CH3 knob” was fused to the N-terminus of the VL of the parental antibody. A GGGS (SEQ ID NO: 17) linker was included between the CH3 domain and the variable region.

In Formats 6-11, the same knob-in-hole CH3 domains were used; Format 6 had no truncation at the C-terminus of the CH3 domains; Format 7 had one amino acid truncation (Δ1) at the C-terminus of the CH3 domains; Format 8 had two amino acids truncation (Δ2) at the C-terminus of the CH3 domains; Format 9 had three amino acids truncation (Δ3) at the C-terminus of the CH3 domains; Format 10 had four amino acids truncation (Δ4) at the C-terminus of the CH3 domains; Format 11 had five acids truncation (Δ5) at the C-terminus of the CH3 domains.

TABLE 6
Tested Antibody Prodrugs

| Format # | Heavy Chain | Light Chain |
|---|---|---|
| 5 | CH3 hole Δ6 + (G₃S)₁ + Parental heavy chain | CH3 knob Δ6 + (G₃S)₁ + Parental light chain |
| 6 | CH3 hole + (G₃S)₁ + Parental heavy chain | CH3 knob + (G₃S)₁ + Parental light chain |
| 7 | CH3 hole Δ1 + (G₃S)₁ + Parental heavy chain | CH3 knob Δ1 + (G₃S)₁ + Parental light chain |
| 8 | CH3 hole Δ2 + (G₃S)₁ + Parental heavy chain | CH3 knob Δ2 + (G₃S)₁ + Parental light chain |
| 9 | CH3 hole Δ3 + (G₃S)₁ + Parental heavy chain | CH3 knob Δ3 + (G₃S)₁ + Parental light chain |
| 10 | CH3 hole Δ4 + (G₃S)₁ + Parental heavy chain | CH3 knob Δ4 + (G₃S)₁ + Parental light chain |
| 11 | CH3 hole Δ5 + (G₃S)₁ + Parental heavy chain | CH3 knob Δ5 + (G₃S)₁ + Parental light chain |

*Tn - n amino acid(s) truncation at the C-terminus of the respective CH3 domain

Format 5 was first compared to Formats 1 and 2 in a cell-based FACS binding assay. As shown in FIG. 5 and Table 8 below, surprisingly, Format 5 had negligible activity as compared to Format 1 (parental antibody) and Format 2 (non-truncated wild-type CH3 with no KIH mutations), suggesting KIH mutations may contribute to the blocking effect of the CH3 masking moiety.

TABLE 7
Protein Sequences of formats 5-11

| | | SEQ ID |
|---|---|---|
| Name | Sequence | NO: |
| CH3 knob | GQPREPQVYTLPPCRDELTKNQVSLWCLVKGFYPSDIAVEWESNGQ | 19 |
| (S354C, T366W) | PENNYKTTPPVLDSDGSFFLYSKLTVDKSRWQQGNVFSCSVMHEAL | |
| | HNHYTQKSLSLSPGK | |
| CH3 hole | GQPREPQVCTLPPSRDELTKNQVSLSCAVKGFYPSDIAVEWESNGQP | 20 |
| (Y349C, T366S, | ENNYKTTPPVLDSDGSFFLVSKLTVDKSRWQQGNVFSCSVMHEALH | |
| L368A, Y407V) | NHYTQKSLSLSPGK | |
| Format 5 heavy | CH3 hole w/ 6 aa truncation (SEQ ID NO: 35) | 21 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSL | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 5 light | CH3 knob w/ 6 aa truncation (SEQ ID NO: 36) | 22 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSL | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRTNGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| Format 6 heavy | CH3 hole (SEQ ID NO: 20) | 23 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSLSLSPGK | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 6 light | CH3 knob (SEQ ID NO: 19) | 24 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLSLSPGK | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRINGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| Format 7 heavy | CH3 hole w/ 1 aa truncation (SEQ ID NO: 37) | 25 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSLSLSPG | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 7 light | CH3 knob w/ 1 aa truncation (SEQ ID NO: 38) | 26 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLSLSPG | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRTNGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| Format 8 heavy | CH3 hole w/ 2 aa truncation (SEQ ID NO: 39) | 27 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSLSLSP | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 8 light | CH3 knob w/ 2 aa truncation (SEQ ID NO: 40) | 28 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLSLSP | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRTNGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| Format 9 heavy | CH3 hole w/ 3 aa truncation (SEQ ID NO: 41) | 29 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSLSLS | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 9 light | CH3 knob w/ 3 aa truncation (SEQ ID NO: 42) | 30 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLSLS | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRINGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| Format 10 heavy | CH3 hole w/ 4 aa truncation (SEQ ID NO: 43) | 31 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSLSL | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 10 light | CH3 knob w/ 4 aa truncation (SEQ ID NO: 44) | 32 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLSL | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRTNGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |
| Format 11 heavy | CH3 hole w/ 5 aa truncation (SEQ ID NO: 45) | 33 |
| chain | GQPREPQVCTLPPSRDELTKNQVSLSCAVKGFYPSDIAVEWESNGQP | |
| | ENNYKTTPPVLDSDGSFFLVSKLTVDKSRWQQGNVFSCSVMHEALH | |
| | NHYTQKSLS | |
| | Linker (SEQ ID NO: 17) | |
| | GGGS | |
| | Cetuximab VH (SEQ ID NO: 1) | |
| | QVQLKQSGPGLVQPSQSLSITCTVSGFSLTNYGVHWVRQSPGKGLE | |
| | WLGVIWSGGNTDYNTPFTSRLSINKDNSKSQVFFKMNSLQSNDTAIY | |
| | YCARALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKV | |
| | DKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAK | |
| | CH3 (SEQ ID NO: 5) | |
| | GQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALH | |
| | NHYTQKSLSLSPGK | |
| Format 11 light | CH3 knob w/ 5 aa truncation (SEQ ID NO: 46) | 34 |
| chain | GQPREPQVYTLPPCRDELTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLS | |
| | Linker (SEQ ID NO: 17) | |
| | GGGS | |
| | Cetuximab VL (SEQ ID NO: 6) | |
| | DILLTQSPVILSVSPGERVSFSCRASQSIGTNIHWYQQRTNGSPRLLIK | |
| | YASESISGIPSRFSGSGSGTDFTLSINSVESEDIADYYCQQNNNWPTTF | |
| | GAGTKLELK | |
| | CLκ (SEQ ID NO: 7) | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLSSPVTKSFNRGEC | |

TABLE 8
Cell-based FACS Results (A431 cells)

| Molecule | EC50 (μg/mL) |
|---|---|
| Isotype (IgG) | — |
| Format 1 | 0.1213 |
| Format 2 | 1.202 |
| Format 5 | — |

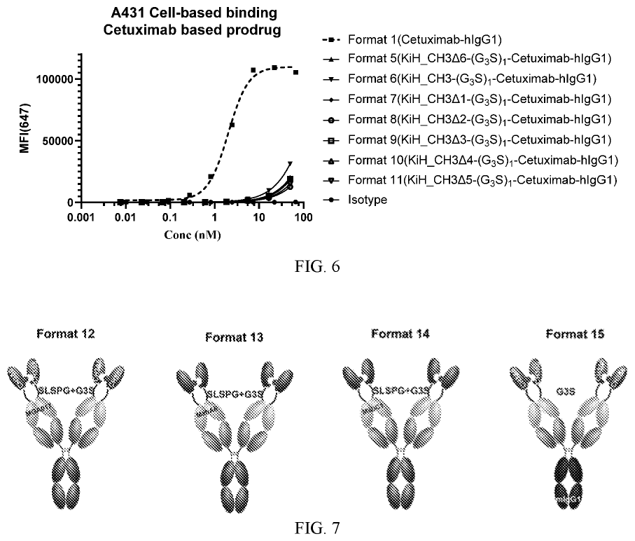

Subsequently, Formats 5-11 were compared to Format 1 (parental antibody) in the cell based FACS binding assay. The results are shown in FIG. 6.

FIG. 6 roughly shows that the shorter the distance was between the CH3 domains and the variable regions, the higher the masking effect the CH3 domains had. Nevertheless, all of Formats 5-11 exhibited excellent blocking effect of the parental antibody's binding activity. The knob-into-hole mutations of masking CH3 is essential for good blocking effect.

According to the results above, new antibody prodrugs referred to as Formats 12-15, were designed as illustrated in FIG. 7, and with sequences shown in Table 9.

Formats 12-14 share the same structure properties as Format 7. All three formats included a KIH mutated CH3A1 as the masking moiety and a GGGS linker connecting the mask and the variable region. The Fc portion is hIgG1. The variable region of Formats 12 was based on the sequence of MGA017, which is an anti-B7-H3 antibody from MacroGenics in clinical stage. The variable region of Formats 13 and 14 were based on the sequence of in-house developed B7-H3 antibodies, MabA6 and MabC1 respectively.

Format 15 was a prodrug based on variable region of MGA017 with KIH mutated CH3Δ6 as the masking moiety and a GGGS (SEQ ID NO: 17) linker. The Fc portion is mIgG1.

TABLE 9
Protein Sequences of Formats 12-15

| | | SEQ ID |
|---|---|---|
| Name | Sequence | NO: |
| Format 12 | Heavy Chain | 73 |
| | CH3Δ1 hole (SEQ ID NO: 37) | |
| | GQPREPQVCTLPPSRDELTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPG | |
| | Linker(SEQ ID NO: 17) | |
| | GGGS | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | CH1 (SEQ ID NO: 2) | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPA | |
| | PIEKTISKAK | |
| | CH3 (SEQ ID NO: 5) | |
| | GQPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQKS | |
| | LSLSPGK | |
| | Light Chain | 74 |
| | CH3Δ1 knob (SEQ ID NO: 38) | |
| | GQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKS | |
| | LSLSPG | |
| | Linker (SEQ ID NO: 17) | |
| | GGGS | |
| | MGA017 VL (SEQ ID NO: 66) | |
| | DIQMTQSPSSLSASVGDRVTITCRASESIYSYLAWYQQKPGKAPKLLVYNT | |
| | KTLPEGVPSRFSGSGSGTDFTLTISSLQPEDFATYYCQHHYGTPPWTFGQGT | |
| | RLEIK | |
| | CLκ (SEQ ID NO: 7) | |
| | RTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKS | |
| | FNRGEC | |
| Format 13 | Heavy Chain | 75 |
| | CH3Δ1 hole (SEQ ID NO: 37) | |
| | GQPREPQVCTLPPSRDELTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPG | |
| | Linker (SEQ ID NO: 17) | |
| | GGGS | |
| | MabA6 VH (SEQ ID NO: 67) | |
| | MGWFYIILFLVATATGVHSQVQLQQPGAELVKPGASVKLSCKASGYTFTS | |
| | YWMHWVNQRPGQGLEWIGMIHPNSGSTNYNEKFKSKATLTVDKSSSTAY | |
| | MQLSSLTSEDSAVYYCARNYGRYFDYWGQGTTLTVSS | |
| | CH1 (SEQ ID NO: 2) | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPA | |
| | PIEKTISKAK | |
| | CH3 (SEQ ID NO: 5) | |
| | GQPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQKS | |
| | LSLSPGK | |
| | Light Chain | 76 |
| | CH3Δ1 knob (SEQ ID NO: 38) | |
| | GQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKS | |
| | LSLSPG | |
| | Linker (SEQ ID NO: 17) | |
| | GGGS | |
| | MabA6 VL (SEQ ID NO: 68) | |
| | MDFQVQIFSFLLISVSVIMSRGQIVLTQSPAIMSASPGEKVTLTCSANSSVSS | |
| | SSLYWYQQKPGSSPKLWIYSTSNLASGVPARFSGSGSGTSYSLTISSMEAED | |
| | AASYFCHQWSSYPLTFGAGTKLELK | |
| | CLκ (SEQ ID NO: 7) | |
| | RTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKS | |
| | FNRGEC | |
| Format 14 | Heavy Chain | |
| | CH3Δ1 hole (SEQ ID NO: 37) | 77 |
| | GQPREPQVCTLPPSRDELTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | SLSPG | |
| | Linker (SEQ ID NO: 17) | |
| | GGGS | |
| | MabC1 VH (SEQ ID NO: 69) | |
| | EVQL VQSGGGL VQPGGSLRLSCAASGFTFSSYAMSWVRQAPGKGLEWVS | |
| | AISGSGGSTYYADSVKGRFTISRDNSKNTLYLQMNSLRAEDTAVYYCAKD | |
| | RYYDILTGTFGYWGQGTLVTVSS | |
| | CH1 (SEQ ID NO: 2) | |
| | ASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPA | |
| | PIEKTISKAK | |
| | CH3 (SEQ ID NO: 5) | |
| | GQPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQKS | |
| | LSLSPGK | |
| | Light Chain | 78 |
| | CH3Δ1 knob (SEQ ID NO: 38) | |
| | GQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKS | |
| | LSLSPG | |
| | Linker (SEQ ID NO: 17) | |
| | GGGS | |
| | MabC1 VL (SEQ ID NO: 70) | |
| | EIVMTQSPDFQSVTPKEKVTITCRASQSIGSSLHWYQQKPDQSPKLLIKHAS | |
| | QPISGVPSRFSGSGSGTVFTLTIQSLEAEDAATYYCQQSSRLPTFGGGTKVEI | |
| | Q | |
| | CLκ (SEQ ID NO: 7) | |
| | RTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKS | |
| | FNRGEC | |
| Format 15 | Heavy Chain | 79 |
| | CH3Δ6 hole (SEQ ID NO: 35) | |
| | GQPREPQVCTLPPSRDELTKNQVSLSCAVKGFYPSDIAVEWESNGQPENNY | |
| | KTTPPVLDSDGSFFLVSKLTVDKSRWQQGNVFSCSVMHEALHNHYTQKSL | |
| | Linker (SEQ ID NO: 17) | |
| | GGGS | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | mIgG1 CH (SEQ ID NO: 71) | |
| | AKTTPPSVYPLAPGSAAQTNSMVTLGCLVKGYFPEPVTVTWNSGSLSSGV | |
| | HTFPAVLQSDLYTLSSSVTVPSSTWPSETVTCNVAHPASSTKVDKKIVPRD | |
| | CGCKPCICTVPEVSSVFIFPPKPKDVLTITLTPKVTCVVVDISKDDPEVQFSW | |
| | FVDDVEVHTAQTQPREEQFNSTFRSVSELPIMHQDWLNGKEFKCRVNSAA | |
| | FPAPIEKTISKTKGRPKAPQVYTIPPPKEQMAKDKVSLTCMITDFFPEDITVE | |
| | WQWNGQPAENYKNTQPIMDTDGSYFVYSKLNVQKSNWEAGNTFTCSVL | |
| | HEGLHNHHTEKSLSHSPGK | |
| | Light Chain | 80 |
| | CH3Δ6 knob (SEQ ID NO: 36) | |
| | GQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKS | |
| | L | |
| | Linker (SEQ ID NO: 17) | |
| | GGGS | |
| | MGA017 VL (SEQ ID NO: 66) | |
| | DIQMTQSPSSLSASVGDRVTITCRASESIYSYLAWYQQKPGKAPKLLVYNT | |
| | KTLPEGVPSRFSGSGSGTDFTLTISSLQPEDFATYYCQHHYGTPPWTFGQGT | |
| | RLEIK | |
| | mouse CLκ (SEQ ID NO: 72) | |
| | RADAAPTVSIFPPSSEQLTSGGASVVCFLNNFYPKDINVKWKIDGSERQNG | |
| | VLNSWTDQDSKDSTYSMSSTLTLTKDEYERHNSYTCEATHKTSTSPIVKSF | |
| | NRNEC | |

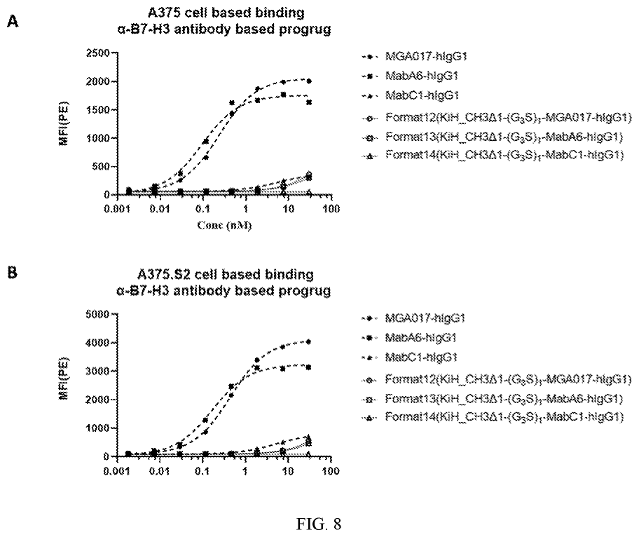

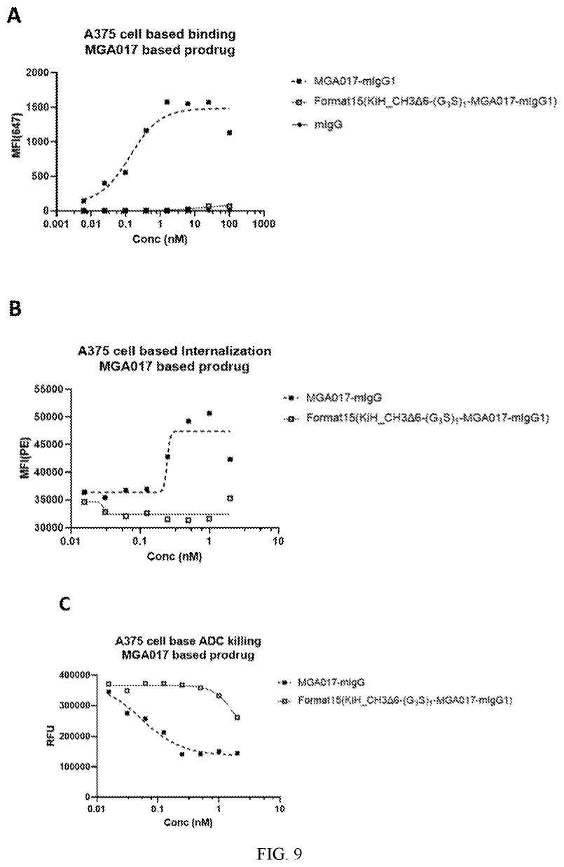

The blocking effect of Formats 12-14 were evaluated in the cell-based binding on B7H3-expressing A375 and A375.S2 cell lines by FACS. As shown in FIG. 8, compared to their parental naked antibodies, Formats 12-14 prodrugs showed significant decrease in the binding activity. As shown in FIG. 9A, Format 15 with the mIgG1 Fc also showed significant decrease in binding potency on A375 cells. These data suggested CH3 KIH masking moiety could efficiently block the blocking activity of different antibodies.

B7-H3 antibodies could be internalized upon binding to the target. To determine whether probody with CH3 masking moiety would also have decreasing internalization, a pHAb thiol Dyes labeled α-mIgG secondary antibody was incubated with Format 15 or its parental antibody MGA017, respectively. The mixture was added into 96-well assay plates pre-seeded A375 cells, and the internalization of the antibody was assessed by measuring fluorescence intensity. As shown in FIG. 9B, the fluorescence signal could not be detected for Format 15, suggesting prodrug with CH3 masking moiety also decrease the internalization of the antibody.

Format 15 or the parental antibody MGA017 was incubated with MMAE labeled α-mIgG secondary antibody and then added into A375 cells. As shown in FIG. 9C, the MMAE mediated cell killing was eliminated for Format 15. These results indicated that probody with CH3 KIH masking moiety could not only diminish the binding activity but also the functional activity of the parental antibody.

### Example 3: Antibody Prodrugs with Linkers of Different Length

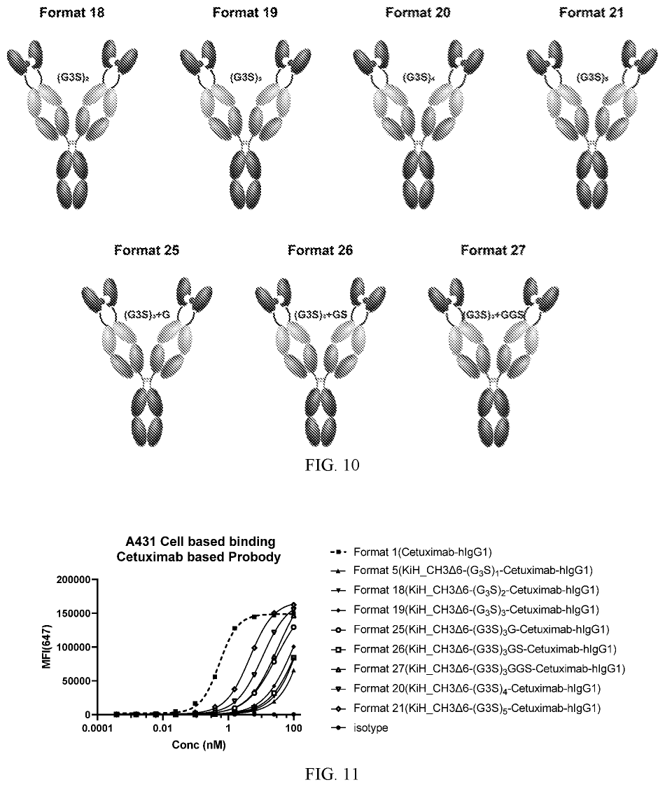

This example described a series of prodrugs with KIH mutated CH346 as the masking moiety and linkers of different lengths ranging from 4 aa to 20 aa. The antibody formats are illustrated in FIG. 10, with sequences shown in Table 10. The variable region of the antibody is based on the sequence of Cetuximab

TABLE 10
Protein Sequences of formats 18-25

| | | SEQ ID |
|---|---|---|
| Name | Sequence | NO: |
| Format 18 | CH3Δ6 hole (SEQ ID NO: 35) | 87 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 18) | |
| | GGGSGGGS | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 18 | CH3Δ6 knob (SEQ ID NO: 36) | 88 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 18) | |
| | GGGSGGGS | |
| | Cetuximab VL (SEQ ID NO: 6) | |
| | DILLTQSPVILSVSPGERVSFSCRASQSIGTNIHWYQQRTNGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |
| Format 19 | CH3Δ6 hole (SEQ ID NO: 35) | 89 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 81) | |
| | GGGSGGGSGGGS | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 19 | CH3Δ6 knob (SEQ ID NO: 36) | 90 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 81) | |
| | GGGSGGGSGGGS | |
| | Cetuximab VL (SEQ ID NO: 6) | |
| | DILLTQSPVILSVSPGERVSFSCRASQSIGTNIHWYQQRTNGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |
| Format 20 | CH3Δ6 hole (SEQ ID NO: 35) | 91 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 85) | |
| | GGGSGGGSGGGSGGGS | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 20 | CH3Δ6 knob (SEQ ID NO: 36) | 92 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 85) | |
| | GGGSGGGSGGGSGGGS | |
| | Cetuximab VL (SEQ ID NO: 6) | |
| | DILLTQSPVILSVSPGERVSFSCRASQSIGTNIHWYQQRTNGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |
| Format 21 | CH3Δ6 hole (SEQ ID NO: 35) | 93 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 86) | |
| | GGGSGGGSGGGSGGGSGGGS | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 21 | CH3Δ6 knob (SEQ ID NO: 36) | 94 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 86) | |
| | GGGSGGGSGGGSGGGSGGGS | |
| | Cetuximab VL (SEQ ID NO: 6) | |
| | DILLTQSPVILSVSPGERVSFSCRASQSIGTNIHWYQQRTNGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |
| Format 25 | CH3Δ6 hole (SEQ ID NO: 35) | 95 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 82) | |
| | GGGSGGGSGGGSG | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 25 | CH3Δ6 knob (SEQ ID NO: 36) | 96 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 82) | |
| | GGGSGGGSGGGSG | |
| | Cetuximab VL (SEQ ID NO: 6) | |
| | DILLTQSPVILSVSPGERVSFSCRASQSIGTNIHWYQQRTNGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |
| Format 26 | CH3Δ6 hole (SEQ ID NO: 35) | 97 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 83) | |
| | GGGSGGGSGGGSGS | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 26 | CH3Δ6 knob (SEQ ID NO: 36) | 98 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 83) | |
| | GGGSGGGSGGGSGS | |
| | Cetuximab VL (SEQ ID NO: 6) | |
| | DILLTQSPVILSVSPGERVSFSCRASQSIGTNIHWYQQRINGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |
| Format 27 | CH3Δ6 hole (SEQ ID NO: 35) | 99 |
| heavy chain | GQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYT | |
| | QKSL | |
| | Linker (SEQ ID NO: 84) | |
| | GGGSGGGSGGGSGGS | |
| | Cetuximab VH (SEQ ID NO: 1) | |
| | QVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCAR | |
| | ALTYYDYEFAYWGQGTLVTVSA | |
| | CH1 (SEQ ID NO: 2) | |
| | ASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAK | |
| | CH3 (SEQ ID NO: 5) | |
| | GQPREPQVYTLPPSREEMTKNQVSLTCLVKGFYPSDIAVEWESNGQPEN | |
| | NYKTTPPVLDSDGSFFLYSKLTVDKSRWQQGNVFSCSVMHEALHNHYT | |
| | QKSLSLSPGK | |
| Format 27 | CH3Δ6 knob (SEQ ID NO: 36) | 100 |
| light chain | GQPREPQVYTLPPCRDELTKNQVSLWCLVKGFYPSDIAVEWESNGQPE | |
| | NNYKTTPPVLDSDGSFFLYSKLTVDKSRWQQGNVFSCSVMHEALHNH | |
| | YTQKSL | |
| | Linker (SEQ ID NO: 84) | |
| | GGGSGGGSGGGSGGS | |
| | Cetuximab VL (SEQ ID NO: 6) | |
| | DILLTQSPVILSVSPGERVSFSCRASQSIGTNIHWYQQRTNGSPRLLIKYA | |
| | SESISGIPSRFSGSGSGTDFTLSINSVESEDIADYYCQQNNNWPTTFGAGT | |
| | KLELK | |
| | CLκ (SEQ ID NO: 7) | |
| | RTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSP | |
| | VTKSFNRGEC | |

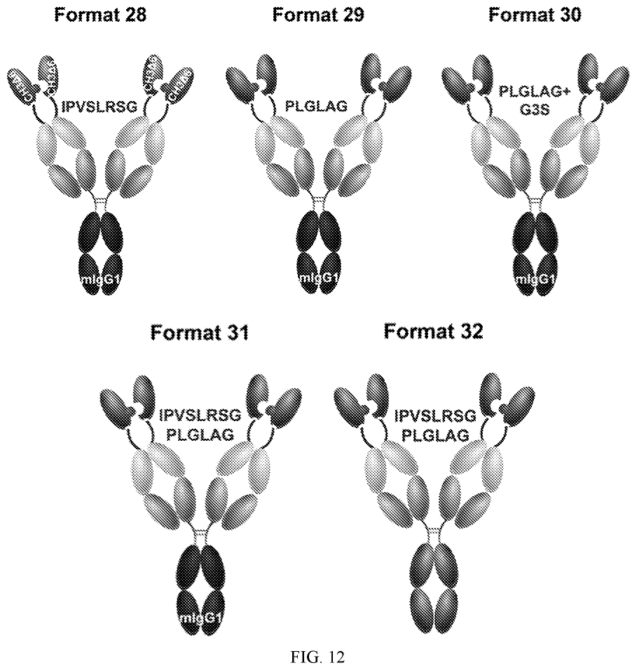

These antibody prodrugs were tested for their binding to human EGFR by FACS. The results are shown in FIG. 11. The blocking activity was reversely correlated to the length of the linker. The linker with the length shorter than 20aa (NOT including 20aa) are optimal for efficient blocking, which leads to at least 20-fold decrease in binding activity.

### Example 4: Antibody Prodrugs with Cleavable Linkers

This example tested the in vitro activities of prodrugs with cleavable linkers. In this example, the cleavable peptide of MMP-2, ‘PLGLAG’ (SEQ ID NO: 55) or ‘IPVSLRSG’ (SEQ ID NO: 64) or the combination of both peptides (IPVSLRSGPLGLAG; SEQ ID NO: 103), were selected as the linker of prodrugs. The variable region of Formats 28-31 are based on the sequence of MGA017, with KIH CH346 as the masking moiety. The antibody design are illustrated in FIG. 12, and sequences were shown in Table 11.

TABLE 11
Protein Sequences of formats 28-32

| | | SEQ ID |
|---|---|---|
| Name | Sequence | NO: |
| Format 28 | CH3Δ6 hole(SEQ ID NO: 35) | 104 |
| heavy chain | GQPREPQVCTLPPSRDELTKNQVSLSCAVKGFYPSDIAVEWESNGQPENN | |
| | YKTTPPVLDSDGSFFLVSKLTVDKSRWQQGNVFSCSVMHEALHNHYTQK | |
| | SL | |
| | Linker(SEQ ID NO: 64) | |
| | IPVSLRSG | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | mIgG1 CH (SEQ ID NO: 71) | |
| | AKTTPPSVYPLAPGSAAQTNSMVTLGCLVKGYFPEPVTVTWNSGSLSSGV | |
| | HTFPAVLQSDLYTLSSSVTVPSSTWPSETVTCNVAHPASSTKVDKKIVPRD | |
| | CGCKPCICTVPEVSSVFIFPPKPKDVLTITLTPKVTCVVVDISKDDPEVQFS | |
| | WFVDDVEVHTAQTQPREEQFNSTFRSVSELPIMHQDWLNGKEFKCRVNS | |
| | AAFPAPIEKTISKTKGRPKAPQVYTIPPPKEQMAKDKVSLTCMITDFFPEDI | |
| | TVEWQWNGQPAENYKNTQPIMDTDGSYFVYSKLNVQKSNWEAGNTFTC | |
| | SVLHEGLHNHHTEKSLSHSPGK | |
| Format 28 | CH3Δ6 knob(SEQ ID NO: 36) | 105 |
| light chain | GQPREPQVYTLPPCRDELTKNQVSLWCLVKGFYPSDIAVEWESNGQPEN | |
| | NYKTTPPVLDSDGSFFLYSKLTVDKSRWQQGNVFSCSVMHEALHNHYTQ | |
| | KSL | |
| | Linker (SEQ ID NO: 64) | |
| | IPVSLRSG | |
| | MGA017 VL (SEQ ID NO: 66) | |
| | DIQMTQSPSSLSASVGDRVTITCRASESIYSYLAWYQQKPGKAPKLLVYN | |
| | TKTLPEGVPSRFSGSGSGTDFTLTISSLQPEDFATYYCQHHYGTPPWTFGQ | |
| | GTRLEIK | |
| | mouse CLκ (SEQ ID NO: 72) | |
| | RADAAPTVSIFPPSSEQLTSGGASVVCFLNNFYPKDINVKWKIDGSERQNG | |
| | VLNSWTDQDSKDSTYSMSSTLTLTKDEYERHNSYTCEATHKTSTSPIVKS | |
| | FNRNEC | |
| Format 29 | CH3Δ6 hole(SEQ ID NO: 35) | 106 |
| heavy chain | GQPREPQVCTLPPSRDELTKNQVSLSCAVKGFYPSDIAVEWESNGQPENN | |
| | YKTTPPVLDSDGSFFLVSKLTVDKSRWQQGNVFSCSVMHEALHNHYTQK | |
| | SL | |
| | Linker (SEQ ID NO: 55) | |
| | PLGLAG | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | mIgG1 CH (SEQ ID NO: 71) | |
| | AKTTPPSVYPLAPGSAAQTNSMVTLGCLVKGYFPEPVTVTWNSGSLSSGV | |
| | HTFPAVLQSDLYTLSSSVTVPSSTWPSETVTCNVAHPASSTKVDKKIVPRD | |
| | CGCKPCICTVPEVSSVFIFPPKPKDVLTITLTPKVTCVVVDISKDDPEVQFS | |
| | WFVDDVEVHTAQTQPREEQFNSTFRSVSELPIMHQDWLNGKEFKCRVNS | |
| | AAFPAPIEKTISKTKGRPKAPQVYTIPPPKEQMAKDKVSLTCMITDFFPEDI | |
| | TVEWQWNGQPAENYKNTQPIMDTDGSYFVYSKLNVQKSNWEAGNTFTC | |
| | SVLHEGLHNHHTEKSLSHSPGK | |
| Format 29 | CH3Δ6 knob (SEQ ID NO: 36) | 107 |
| light chain | GQPREPQVYTLPPCRDELTKNQVSLWCLVKGFYPSDIAVEWESNGQPEN | |
| | NYKTTPPVLDSDGSFFLYSKLTVDKSRWQQGNVFSCSVMHEALHNHYTQ | |
| | KSL | |
| | Linker (SEQ ID NO: 55) | |
| | PLGLAG | |
| | MGA017 VL (SEQ ID NO: 66) | |
| | DIQMTQSPSSLSASVGDRVTITCRASESIYSYLAWYQQKPGKAPKLLVYN | |
| | TKTLPEGVPSRFSGSGSGTDFTLTISSLQPEDFATYYCQHHYGTPPWTFGQ | |
| | GTRLEIK | |
| | mouse CLκ (SEQ ID NO: 72) | |
| | RADAAPTVSIFPPSSEQLTSGGASVVCFLNNFYPKDINVKWKIDGSERQNG | |
| | VLNSWTDQDSKDSTYSMSSTLTLTKDEYERHNSYTCEATHKTSTSPIVKS | |
| | FNRNEC | |
| Format 30 | CH3Δ6 hole (SEQ ID NO: 35) | 108 |
| heavy chain | GQPREPQVCTLPPSRDELTKNQVSLSCAVKGFYPSDIAVEWESNGQPENN | |
| | YKTTPPVLDSDGSFFLVSKLTVDKSRWQQGNVFSCSVMHEALHNHYTQK | |
| | SL | |
| | Linker (SEQ ID NO: 102) | |
| | PLGLAGGGGS | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | mIgG1 CH (SEQ ID NO: 71) | |
| | AKTTPPSVYPLAPGSAAQTNSMVTLGCLVKGYFPEPVTVTWNSGSLSSGV | |
| | HTFPAVLQSDLYTLSSSVTVPSSTWPSETVTCNVAHPASSTKVDKKIVPRD | |
| | CGCKPCICTVPEVSSVFIFPPKPKDVLTITLTPKVTCVVVDISKDDPEVQFS | |
| | WFVDDVEVHTAQTQPREEQFNSTFRSVSELPIMHQDWLNGKEFKCRVNS | |
| | AAFPAPIEKTISKTKGRPKAPQVYTIPPPKEQMAKDKVSLTCMITDFFPEDI | |
| | TVEWQWNGQPAENYKNTQPIMDTDGSYFVYSKLNVQKSNWEAGNTFTC | |
| | SVLHEGLHNHHTEKSLSHSPGK | |
| Format 30 | CH3Δ6 knob (SEQ ID NO: 36) | 109 |
| light chain | GQPREPQVYTLPPCRDELTKNQVSLWCLVKGFYPSDIAVEWESNGQPEN | |
| | NYKTTPPVLDSDGSFFLYSKLTVDKSRWQQGNVFSCSVMHEALHNHYTQ | |
| | KSL | |
| | Linker (SEQ ID NO: 102) | |
| | PLGLAGGGGS | |
| | MGA017 VL (SEQ ID NO: 66) | |
| | DIQMTQSPSSLSASVGDRVTITCRASESIYSYLAWYQQKPGKAPKLLVYN | |
| | TKTLPEGVPSRFSGSGSGTDFTLTISSLQPEDFATYYCQHHYGTPPWTFGQ | |
| | GTRLEIK | |
| | mouse CLκ (SEQ ID NO: 72) | |
| | RADAAPTVSIFPPSSEQLTSGGASVVCFLNNFYPKDINVKWKIDGSERQNG | |
| | VLNSWTDQDSKDSTYSMSSTLTLTKDEYERHNSYTCEATHKTSTSPIVKS | |
| | FNRNEC | |
| Format 31 | CH3Δ6 hole (SEQ ID NO: 35) | 110 |
| heavy chain | GQPREPQVCTLPPSRDELTKNQVSLSCAVKGFYPSDIAVEWESNGQPENN | |
| | YKTTPPVLDSDGSFFLVSKLTVDKSRWQQGNVFSCSVMHEALHNHYTQK | |
| | SL | |
| | Linker (SEQ ID NO: 103) | |
| | IPVSLRSGPLGLAG | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | mlgG1 CH (SEQ ID NO: 71) | |
| | AKTTPPSVYPLAPGSAAQTNSMVTLGCLVKGYFPEPVTVTWNSGSLSSGV | |
| | HTFPAVLQSDLYTLSSSVTVPSSTWPSETVTCNVAHPASSTKVDKKIVPRD | |
| | CGCKPCICTVPEVSSVFIFPPKPKDVLTITLTPKVTCVVVDISKDDPEVQFS | |
| | WFVDDVEVHTAQTQPREEQFNSTFRSVSELPIMHQDWLNGKEFKCRVNS | |
| | AAFPAPIEKTISKTKGRPKAPQVYTIPPPKEQMAKDKVSLTCMITDFFPEDI | |
| | TVEWQWNGQPAENYKNTQPIMDTDGSYFVYSKLNVQKSNWEAGNTFTC | |
| | SVLHEGLHNHHTEKSLSHSPGK | |
| Format 31 | CH3Δ6 knob (SEQ ID NO: 36) | 111 |
| light chain | GQPREPQVYTLPPCRDELTKNQVSLWCLVKGFYPSDIAVEWESNGQPEN | |
| | NYKTTPPVLDSDGSFFLYSKLTVDKSRWQQGNVFSCSVMHEALHNHYTQ | |
| | KSL | |
| | Linker (SEQ ID NO: 103) | |
| | IPVSLRSGPLGLAG | |
| | MGA017 VL (SEQ ID NO: 66) | |
| | DIQMTQSPSSLSASVGDRVTITCRASESIYSYLAWYQQKPGKAPKLLVYN | |
| | TKTLPEGVPSRFSGSGSGTDFTLTISSLQPEDFATYYCQHHYGTPPWTFGQ | |
| | GTRLEIK | |
| | mouse CLκ (SEQ ID NO: 72) | |
| | RADAAPTVSIFPPSSEQLTSGGASVVCFLNNFYPKDINVKWKIDGSERQNG | |
| | VLNSWTDQDSKDSTYSMSSTLTLTKDEYERHNSYTCEATHKTSTSPIVKS | |
| | FNRNEC | |
| Format 32 | CH3Δ6 hole (SEQ ID NO: 35) | 112 |
| heavy chain | GQPREPQVCTLPPSRDELTKNQVSLSCAVKGFYPSDIAVEWESNGQPENN | |
| | YKTTPPVLDSDGSFFLVSKLTVDKSRWQQGNVFSCSVMHEALHNHYTQK | |
| | SL | |
| | Linker (SEQ ID NO: 103) | |
| | IPVSLRSGPLGLAG | |
| | MGA017 VH (SEQ ID NO: 65) | |
| | EVQLVESGGGLVKPGGSLRLSCAASGFTFSSYGMSWVRQAPGKGLEWVA | |
| | TINSGGSNTYYPDSLKGRFTISRDNAKNSLYLQMNSLRAEDTAVYYCARH | |
| | DGGAMDYWGQGTTVTVSS | |
| | CH1 (SEQ ID NO: 2) | |
| | ASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKAL | |
| | PAPIEKTISKAK | |
| | CH3 (SEQ ID NO: 5) | |
| | GQPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQK | |
| | SLSLSPGK | |
| Format 32 | CH3Δ6 knob (SEQ ID NO: 36) | 113 |
| light chain | GQPREPQVYTLPPCRDELTKNQVSLWCLVKGFYPSDIAVEWESNGQPEN | |
| | NYKTTPPVLDSDGSFFLYSKLTVDKSRWQQGNVFSCSVMHEALHNHYTQ | |
| | KSL | |
| | Linker (SEQ ID NO: 103) | |
| | IPVSLRSGPLGLAG | |
| | MGA017 VL (SEQ ID NO: 66) | |
| | DIQMTQSPSSLSASVGDRVTITCRASESIYSYLAWYQQKPGKAPKLLVYN | |
| | TKTLPEGVPSRFSGSGSGTDFTLTISSLQPEDFATYYCQHHYGTPPWTFGQ | |
| | GTRLEIK | |
| | CLκ (SEQ ID NO: 7) | |
| | RTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTK | |
| | SFNRGEC | |

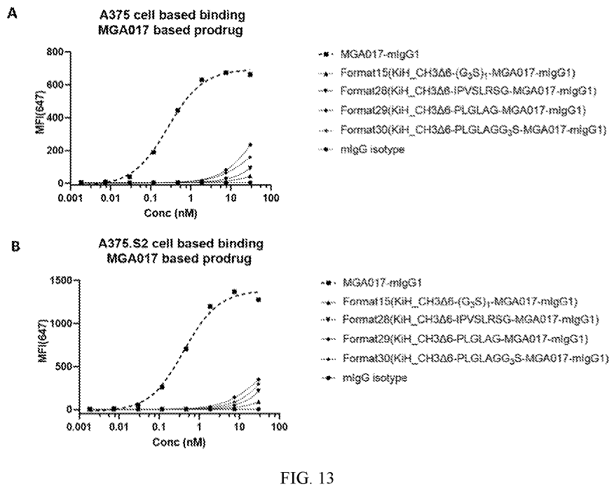

As shown in FIG. 13, compared to their parental antibody MGA017, prodrugs format 28-30 with cleavable linkers also showed excellent blocking effect in cell-based binding on A375 cells and A375.S2 cells.

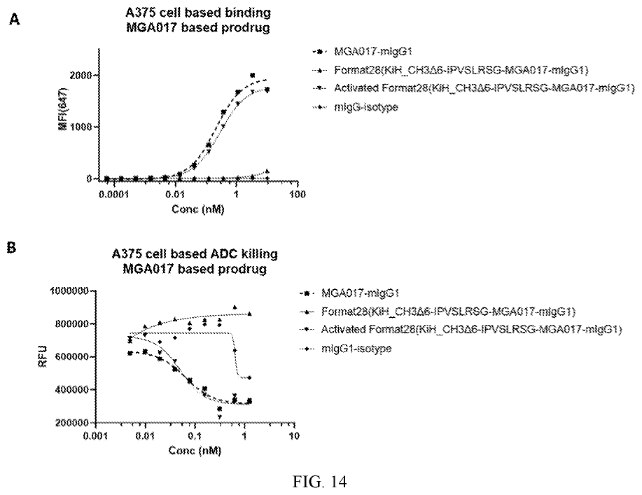

In vitro protease activation assay was performed to determine whether the function of prodrug antibody could be restored after enzymatic cleavage of the masking moiety. Format 28 with linker ‘IPVSLRSG’ (SEQ ID NO: 64) was enzymatically activated by addition of MMP-2. As shown in FIG. 14A, activated Format 28 showed comparable binding activity to the parental antibody (MGA017-mIgG1). To determine whether the function of the prodrug could be restored as well, tumor killing mediated by toxic payload MMAE was performed. As presented in FIG. 14B, MMAE conjugated prodrug format 28 showed no killing effect on A375, while in the same experiment setting, enzymatically activated format 28 showed comparable tumor killing, compared to the parental MGA017. These data suggested that the function of prodrug with CH3-KIH moiety and cleavable linker could be restored when the masking moiety was properly removed by the enzyme.

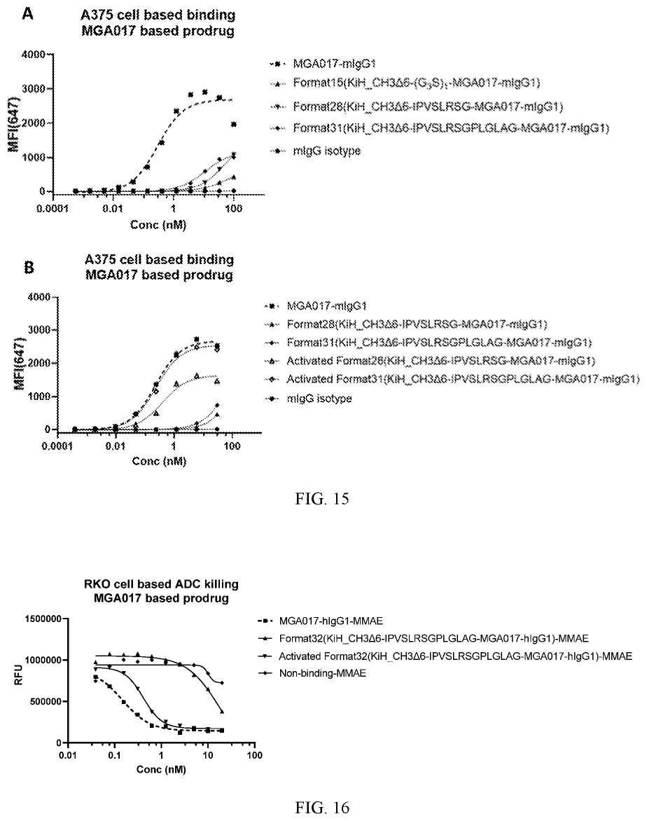

Format 31 was a prodrug that has the same design with Format 28, except the linker was replaced by ‘IPVSLRSGPLGLAG’ (SEQ ID NO: 103), a combination of two MMP-2 cleavage sites. As shown in FIG. 15A, compared to the naked antibody MGA017, Format 31 still showed good blocking effect on cell-based binding assay. To compare the proteolytical efficiency of one cleavage site and two cleavage sites under same experimental condition, Format 28 and Format 31 were mixed with a subsaturated amount of MMP-2 and activated antibodies were evaluated in the cell based assay against the B7H3-expressing A375. As shown in FIG. 15B, activated Format 31 showed comparable binding to MGA017-mIgG1, while proteolytically activated Format 28 also partially restored the binding activity but in a less extent than activated Format 31. These results suggested that Format 31 with two tandem cleavage sites showed obviously better proteolysis efficacy than Format 28 with one cleavage site.

Format 32 was similar to Format 31, except the Fc portion is human IgG1. To determine whether the function of probodies with two cleavage sites could be restored after proteolysis by MMP2, probody Format 32 was conjugated with MMAE and then cleaved by in vitro addition of MMP-2. As shown in FIG. 16, Format 31-conjugated with MMAE showed negligible killing, similar to non-binding-MMAE (an MMAE labeled anti-HEL hIgG1), while activated Format 31 showed a strong killing effect which is compared to the MGA017-MMAE. These results supported that CH3 masking prodrug could be cleaved and the function could be restored after proteolysis by a certain protease.

### Example 5: Antibody Prodrugs with Other Immunoglobin Domains as Mask

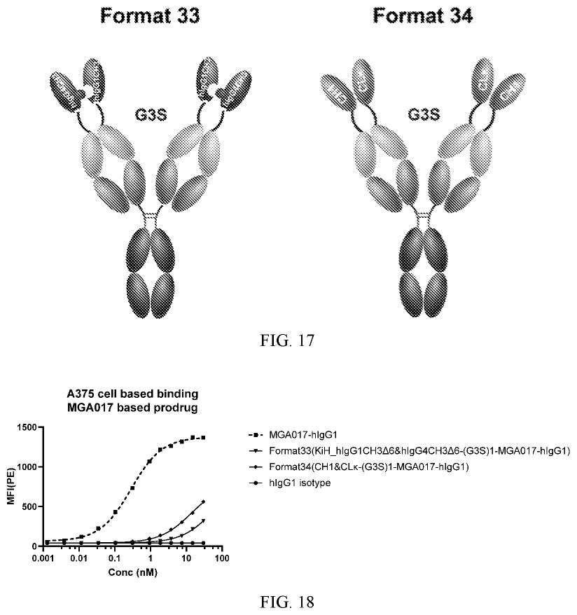

This example described prodrugs with other immunoglobin domains as potential masking moieties. Format 33 included a pair of human IgG1 CH3 and human IgG4 fragments as the mask and GGGS (SEQ ID NO: 17) linker. Format 34 included a pair of human IgG1 CH1 (where IgG1 CH1 means CH1 plus EPKSC (SEQ ID NO: 120)) and human CLK fragments as the mask and GGGS (SEQ ID NO: 17) linker. These prodrugs are illustrated in FIG. 17 and sequences were shown in Table 12. The binding of these prodrugs against B7H3-expressing A375 cell line were tested. As shown in FIG. 18, Formats 33 and 34 showed obvious blocking effects, compared to un-masked MGA017. In general, however, the blocking effects of the constant regions on Format 34 were weaker than that of Format 33 or 12, demonstrating the higher blocking efficacy of CH3/CH3 pairs.

TABLE 12
Protein Sequences of formats 33-34

| Name | Sequence | SEQ ID NO: |
|---|---|---|
| Format 33 | IgG1 CH3Δ6 hole (SEQ ID NO: 35) | 116 |
| heavy chain | GQPREPQVCTLPPSRDELTKNQVSLSCAVKGFYPSDIAVEWES | |
| | NGQPENNYKTTPPVLDSDGSFFLVSKLTVDKSRWQQGNVFSC | |
| | SVMHEALHNHYTQKSL | |
| | Linker (SEQ ID NO: 17) | |
| | GGGS | |
| | VH (SEQ ID NO: 65) | |
| | EVQLVESGGGLVKPGGSLRLSCAASGFTFSSYGMSWVRQAP | |
| | GKGLEWVATINSGGSNTYYPDSLKGRFTISRDNAKNSLYLQM | |
| | NSLRAEDTAVYYCARHDGGAMDYWGQGTTVTVSS | |
| | CH1 (SEQ ID NO: 2) | |
| | ASTKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEV | |
| | KFNWYVDGVEVHNAKTKPREEQYNSTYRVVSVLTVLHQDW | |
| | LNGKEYKCKVSNKALPAPIEKTISKAK | |
| | CH3 (SEQ ID NO: 5) | |
| | GQPREPQVYTLPPSREEMTKNQVSLTCLVKGFYPSDIAVEWE | |
| | SNGQPENNYKTTPPVLDSDGSFFLYSKLTVDKSRWQQGNVFS | |
| | CSVMHEALHNHYTQKSLSLSPGK | |
| Format 33 | IgG4 CH3Δ6 knob (SEQ ID NO: 114) | 117 |
| light chain | GQPREPQVYTLPPCQEEMTKNQVSLWCLVKGFYPSDIAVEW | |
| | ESNGQPENNYKTTPPVLDSDGSFFLYSRLTVDKSRWQEGNVF | |
| | SCSVMHEALHNHYTQKSL | |
| | Linker (SEQ ID NO: 17) | |
| | GGGS | |
| | VL (SEQ ID NO: 66) | |
| | DIQMTQSPSSLSASVGDRVTITCRASESIYSYLAWYQQKPGKA | |
| | PKLLVYNTKTLPEGVPSRFSGSGSGTDFTLTISSLQPEDFATYY | |
| | CQHHYGTPPWTFGQGTRLEIK | |
| | CLκ (SEQ ID NO: 7) | |
| | RTVAAPSVFIFPPSDEQLKSGTASVVCLLNNFYPREAKVQWK | |
| | VDNALQSGNSQESVTEQDSKDSTYSLSSTLTLSKADYEKHKV | |
| | YACEVTHQGLSSPVTKSFNRGEC | |
| Format 34 | CLκ (SEQ ID NO: 7) | 118 |
| heavy chain | RTVAAPSVFIFPPSDEQLKSGTASVVCLLNNFYPREAKVQWK | |
| | VDNALQSGNSQESVTEQDSKDSTYSLSSTLTLSKADYEKHKV | |
| | YACEVTHQGLSSPVTKSFNRGEC | |
| | Linker(SEQ ID NO: 17) | |
| | GGGS | |
| | VH (SEQ ID NO: 65) | |
| | EVQLVESGGGLVKPGGSLRLSCAASGFTFSSYGMSWVRQAP | |
| | GKGLEWVATINSGGSNTYYPDSLKGRFTISRDNAKNSLYLQM | |
| | NSLRAEDTAVYYCARHDGGAMDYWGQGTTVTVSS | |
| | CH1 (SEQ ID NO: 2) | |
| | ASTKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKRV | |
| | Hinge (SEQ ID NO: 3) | |
| | EPKSCDKTHTCPPCP | |
| | CH2 (SEQ ID NO: 4) | |
| | APELLGGPSVFLFPPKPKDTLMISRTPEVTCVVVDVSHEDPEV | |
| | KFNWYVDGVEVHNAKTKPREEQYNSTYRVVSVLTVLHQDW | |
| | LNGKEYKCKVSNKALPAPIEKTISKAK | |
| | CH3 (SEQ ID NO: 5) | |
| | GQPREPQVYTLPPSREEMTKNQVSLTCLVKGFYPSDIAVEWE | |
| | SNGQPENNYKTTPPVLDSDGSFFLYSKLTVDKSRWQQGNVFS | |
| | CSVMHEALHNHYTQKSLSLSPGK | |
| Format 34 | CH1 plus (SEQ ID NO: 115) | 119 |
| light chain | ASTKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKKVEPKSC | |
| | Linker (SEQ ID NO: 7) | |
| | GGGS | |
| | VL (SEQ ID NO: 66) | |
| | DIQMTQSPSSLSASVGDRVTITCRASESIYSYLAWYQQKPGKA | |
| | PKLLVYNTKTLPEGVPSRFSGSGSGTDFTLTISSLQPEDFATYY | |
| | CQHHYGTPPWTFGQGTRLEIK | |
| | CLκ (SEQ ID NO: 7) | |
| | RTVAAPSVFIFPPSDEQLKSGTASVVCLLNNFYPREAKVQWK | |
| | VDNALQSGNSQESVTEQDSKDSTYSLSSTLTLSKADYEKHKV | |
| | YACEVTHQGLSSPVTKSFNRGEC | |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A molecule comprising (a) an immunoglobulin superfamily constant region or a fragment thereof covalently coupled to (b) an immunoglobulin superfamily variable region, wherein the variable region, when not coupled to the constant region, can bind to a target molecule, but the coupling of the constant region to the variable region inhibits such binding.

2. The molecule of claim 1, wherein the constant region (a) is fused to the N-terminus of the variable region or (b) is conjugated to the variable region.

3. The molecule of claim 1, which does not include an additional immunoglobulin superfamily variable region on the N-terminal side of the immunoglobulin superfamily constant region.

4. The molecule of claim 1, wherein the constant region is selected from the group consisting of an IgG CH3, IgG CH2, IgG CH1, IgG CL, and a T-cell receptor (TCR) constant region, preferably CH3.

5. The molecule of claim 1, wherein the variable region is selected from the group consisting of heavy chain variable region (VH), a light chain variable region (VL), and a T-cell receptor (TCR) variable region.

6. The molecule of claim 1, wherein the constant region is fused to the N-terminus of the variable region.

7. The molecule of claim 6, which comprises a heavy chain variable region (VH), a first immunoglobulin superfamily constant region fused to the N-terminus of the VH, a light chain variable region (VL), and a second immunoglobulin superfamily constant region fused to the N-terminus of the VL, wherein the VH and VL collectively have binding specificity to the target molecule, and the first and second constant regions pair with each other.

8. The molecule of claim 7, wherein the first and second constant regions are two CH3, a CH1 and a CL, or a TCR alpha chain and a TCR beta chain.

9. The molecule of claim 8, wherein the two constant regions are modified, as compared to the wild-type constant regions, to increase the heterodimerization of the masking moiety.

10. The molecule of claim 9, wherein the two constant regions are modified, as compared to the wild-type constant regions, to include knob-in-hole substitutions, or charge-pair substitutions.

11-12. (canceled)

13. The molecule of claim 6, which comprises:
a first antigen-binding unit comprising a first VH paired to a first VL,
a second antigen-binding unit comprising a second VH paired to a second VL,
a first immunoglobulin superfamily constant region fused to the N-terminus of the first VH, a second immunoglobulin superfamily constant region fused to the N-terminus of the first VL, a third immunoglobulin superfamily constant region fused to the N-terminus of the second VH, and a fourth immunoglobulin superfamily constant region fused to the N-terminus of the second VL,
wherein the first immunoglobulin superfamily constant region pairs with the second immunoglobulin superfamily constant region and inhibits the binding of the first antigen-binding unit, and the third immunoglobulin superfamily constant region pairs with the fourth immunoglobulin superfamily constant region and inhibits the binding of the second antigen-binding unit.

14. The molecule of claim 13, wherein the first antigen-binding unit and the second antigen-binding unit are the same or different.

15. (canceled)

16. (canceled)

17. The molecule of claim 6, wherein there are no more than 23 amino acid residues, preferably no more than 22, 21 or 20 amino acid residues, and more preferably no more than 15, 14, 13, 12, 11, 10, 9, or 8 amino acid residues between T437, according to EU numbering (T468 according to Kabat numbering), of each CH3 domain and the N-terminus of the corresponding variable region.

18-30. (canceled)

31. The molecule of claim 4, wherein each CH3 domain comprises amino acid residues G371 through T437, according to EU numbering, of a full-length CH3 domain.

32. The molecule of claim 4, wherein each CH3 domain comprises amino acid residues K360 through T437, according to EU numbering, of a full-length CH3 domain.

33. The molecule of claim 4, wherein each CH3 domain comprises amino acid residues E345 through T437, according to EU numbering, of a full-length CH3 domain.

34. The molecule of claim 4, wherein each CH3 domain comprises amino acid residues 31-97 of SEQ ID NO:10, or amino acid residues 20-97, 10-97, 5-97, 4-97, 3-97, 2-97, or 5-101 of SEQ ID NO:10.

35. The molecule of claim 34, wherein one of the CH3 domains comprises amino acid residues 1-97 of SEQ ID NO:19 and the other CH3 domain comprises amino acid residues 1-97 of SEQ ID NO:20.

36-37. (canceled)

38. A fusion protein comprising a cleavable peptide linker fused to the C-terminus of an immunoglobulin superfamily constant region, wherein the fusion protein does not include an antigen-binding fragment on the N-terminal side of the immunoglobulin superfamily constant region.

39-48. (canceled)

49. A method for delivering an active antibody or antigen-binding fragment to a subject, comprising administering to the subject the molecule of claim 1, wherein the cleavable linker is cleaved in the subject thereby releasing the antibody or antigen-binding fragment in the subject.

50. (canceled)


SEQUENCE LISTING (120 sequences)

SEQ ID NO: 1 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 2 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 15 aa, synthetic construct
EPKSCDKTHTCPPCP

SEQ ID NO: 4 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 5 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 7 - protein, 107 aa, synthetic construct [sequence omitted]

SEQ ID NO: 8 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 556 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 321 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 560 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 325 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 564 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 329 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 4 aa, synthetic construct
GGGS

SEQ ID NO: 18 - protein, 8 aa, synthetic construct
GGGSGGGS

SEQ ID NO: 19 - protein, 107 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 107 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 554 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 319 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 560 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 325 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 559 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 324 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 558 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 323 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 557 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 322 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 556 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 321 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 555 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 320 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 101 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSL

SEQ ID NO: 36 - protein, 101 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSL

SEQ ID NO: 37 - protein, 106 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 38 - protein, 106 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 105 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 40 - protein, 105 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 41 - protein, 104 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLS

SEQ ID NO: 42 - protein, 104 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLS

SEQ ID NO: 43 - protein, 103 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSL

SEQ ID NO: 44 - protein, 103 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSL

SEQ ID NO: 45 - protein, 102 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLS

SEQ ID NO: 46 - protein, 102 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLS

SEQ ID NO: 47 - protein, 107 aa, synthetic construct; identical to SEQ ID NO: 5

SEQ ID NO: 48 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 49 - protein, 107 aa, synthetic construct
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 50 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 8 aa, synthetic construct
LSGRSDNH

SEQ ID NO: 52 - protein, 4 aa, synthetic construct
LSGK

SEQ ID NO: 53 - skipped

SEQ ID NO: 54 - protein, 7 aa, synthetic construct
VPLSLYS

SEQ ID NO: 55 - protein, 6 aa, synthetic construct
PLGLAG

SEQ ID NO: 56 - protein, 10 aa, synthetic construct
VLVPMAMMAS

SEQ ID NO: 57 - skipped

SEQ ID NO: 58 - protein, 4 aa, synthetic construct
AGPR

SEQ ID NO: 59 - protein, 4 aa, synthetic construct
AANL

SEQ ID NO: 60 - protein, 4 aa, synthetic construct
PTNL

SEQ ID NO: 61 - protein, 8 aa, synthetic construct
TSGRSANP

SEQ ID NO: 62-63 - skipped (2 entries)

SEQ ID NO: 64 - protein, 8 aa, synthetic construct
IPVSLRSG

SEQ ID NO: 65 - protein, 117 aa, synthetic construct
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 66 - protein, 108 aa, synthetic construct
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 67 - protein, 136 aa, synthetic construct
MGWFYIILFLVATATGVHSQVQLQQPGAELVKPGASVKLSCKASGYTFTSYWMHWVNQRPGQGLEWIGMIHPNSGSTNYNEKFKSKATLTVDKSSSTAYMQLSSLTSEDSAVYYCARNYGRYFDYWGQGTTLTVSS

SEQ ID NO: 68 - protein, 130 aa, synthetic construct
MDFQVQIFSFLLISVSVIMSRGQIVLTQSPAIMSASPGEKVTLTCSANSSVSSSSLYWYQQKPGSSPKLWIYSTSNLASGVPARFSGSGSGTSYSLTISSMEAEDAASYFCHQWSSYPLTFGAGTKLELK

SEQ ID NO: 69 - protein, 122 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDRYYDILTGTFGYWGQGTLVTVSS

SEQ ID NO: 70 - protein, 106 aa, synthetic construct
EIVMTQSPDFQSVTPKEKVTITCRASQSIGSSLHWYQQKPDQSPKLLIKHASQPISGVPSRFSGSGSGTVFTLTIQSLEAEDAATYYCQQSSRLPTFGGGTKVEIQ

SEQ ID NO: 71 - protein, 324 aa, synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 72 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 73 - protein, 557 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 325 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 576 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSMGWFYIILFLVATATGVHSQVQLQQPGAELVKPGASVKLSCKASGYTFTSYWMHWVNQRPGQGLEWIGMIHPNSGSTNYNEKFKSKATLTVDKSSSTAYMQLSSLTSEDSAVYYCARNYGRYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 347 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSMDFQVQIFSFLLISVSVIMSRGQIVLTQSPAIMSASPGEKVTLTCSANSSVSSSSLYWYQQKPGSSPKLWIYSTSNLASGVPARFSGSGSGTSYSLTISSMEAEDAASYFCHQWSSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 562 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSEVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDRYYDILTGTFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 323 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSEIVMTQSPDFQSVTPKEKVTITCRASQSIGSSLHWYQQKPDQSPKLLIKHASQPISGVPSRFSGSGSGTVFTLTIQSLEAEDAATYYCQQSSRLPTFGGGTKVEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 546 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 80 - protein, 320 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 81 - protein, 12 aa, synthetic construct
GGGSGGGSGGGS

SEQ ID NO: 82 - protein, 13 aa, synthetic construct
GGGSGGGSGGGSG

SEQ ID NO: 83 - protein, 14 aa, synthetic construct
GGGSGGGSGGGSGS

SEQ ID NO: 84 - protein, 15 aa, synthetic construct
GGGSGGGSGGGSGGS

SEQ ID NO: 85 - protein, 16 aa, synthetic construct
GGGSGGGSGGGSGGGS

SEQ ID NO: 86 - protein, 20 aa, synthetic construct
GGGSGGGSGGGSGGGSGGGS

SEQ ID NO: 87 - protein, 558 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 323 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 562 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 327 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 566 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 331 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSGGGSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 570 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSGGGSGGGSGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 335 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSGGGSGGGSGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 563 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSGGGSGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 328 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSGGGSGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 564 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSGGGSGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 329 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSGGGSGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 565 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSGGGSGGGSGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 330 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLGGGSGGGSGGGSGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 9 aa, synthetic construct
PLGLAGGGS

SEQ ID NO: 102 - protein, 10 aa, synthetic construct
PLGLAGGGGS

SEQ ID NO: 103 - protein, 14 aa, synthetic construct
IPVSLRSGPLGLAG

SEQ ID NO: 104 - protein, 550 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLIPVSLRSGEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 105 - protein, 324 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLIPVSLRSGDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 106 - protein, 548 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLPLGLAGEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 107 - protein, 322 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLPLGLAGDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 108 - protein, 552 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLPLGLAGGGGSEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 109 - protein, 326 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLPLGLAGGGGSDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 110 - protein, 556 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLIPVSLRSGPLGLAGEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 111 - protein, 330 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLIPVSLRSGPLGLAGDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 112 - protein, 562 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLIPVSLRSGPLGLAGEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 330 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLIPVSLRSGPLGLAGDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 101 aa, synthetic construct
GQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSL

SEQ ID NO: 115 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 116 - protein, 552 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLGGGSEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 320 aa, synthetic construct
GQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLGGGSDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 558 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSEVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 322 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSDIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 5 aa, synthetic construct
EPKSC